(19) United States
(12) Patent Application Publication
(10) Pub. No.: US 2026/0009074 A1
(43) Pub. Date: Jan. 8, 2026

(54) ARTIFICIAL SMALL RNA SPIKE-IN COCKTAIL FOR PROCESS CONTROL AND NORMALIZATION

(71) Applicant: HUMMINGBIRD DIAGNOSTICS GmbH, Heidelberg (DE)

(72) Inventors: Bruno Steinkraus, Hanstedt (DE); Rastislav Horos, Heidelberg (DE); Andrej-Nikolai Spiess, Hanstedt (DE); Tobias Sikosek, Heidelberg (DE)

(21) Appl. No.: 18/993,516

(22) PCT Filed: Jul. 3, 2023
(86) PCT No.: PCT/EP2023/068275

§ 371 Date: Jan. 10, 2025

(30) Foreign Application Priority Data
Jul. 20, 2022 (EP) 22186130.5

(51) Int. Cl. C12Q 1/6876

(52) U.S. Cl. CPC C12Q 1/6876; C12Q 2600/158; C12Q 2600/178

(57) ABSTRACT

The present invention relates to a composition which can be used as spike-in cocktail. Further, the present invention relates to a kit comprising the composition. Furthermore, the present invention relates to a method for examining a sample, wherein the composition is used.

### CROSS REFERENCE TO RELATED APPLICATIONS

This application is a U.S. National Stage of PCT/EP2023/068275, International Filing Date Jul. 3, 2023, which claims priority to European Application No. 22186130.5, filing date Jul. 20, 2022.

### REFERENCE TO SEQUENCE LISTING IN ELECTRONIC FORMAT

The contents of the electronic sequence listing (505-78-pct-sequence-listing.xml; Size: 65,780 bytes; and Date of Creation: Jan. 10, 2025) is herein incorporated by reference in its entirety.

The present invention relates to a composition which can be used as spike-in cocktail. Further, the present invention relates to a kit comprising the composition. Furthermore, the present invention relates to a method for examining a sample, wherein the composition is used.

### BACKGROUND OF THE INVENTION

RNAs such as small RNAs in biological samples like biofluids play an important role as prognostic and diagnostic biomarkers for many human disease states. However, accurate analysis of these RNAs in biological samples such as biofluids is of great importance with regard to the meaningfulness of these data in the biomarker field.

The analysis of RNA expression profiles involves several intricate steps. (1) RNA, including small RNA, must be extracted from a biological source, e.g. blood or saliva, without distorting the original relative abundances of the RNA (linearity must be preserved). (2) The small RNA abundances must be reliably quantified. Since both steps can inconsistently introduce bias (e.g. through the presence of inhibitors of cDNA synthesis) it is crucial to standardize but also monitor both the efficiency of RNA extraction and detection. Addressing these challenges can be achieved in several ways.

Firstly, performing RNA integrity analysis of endogenous RNAs can be used for quality control by comparing electrophoretic RNA profiles to expected ones on the basis of previously analysed samples e.g. small RNA bioanalyzer traces. However, such a global analysis only provides a bird's eye view of the sample integrity and lacks granularity.

Secondly, it is possible to define reference values of endogenous small RNAs, e.g. housekeeping genes belonging to nuclear or splicing RNAs, that can serve as yardsticks against which newly measured samples are compared. Samples with housekeeping genes falling outside expected ranges could, thus, be disqualified from analysis as outliers. However, using endogenous features of a sample for quality control makes it impossible to disentangle samples which underwent a technically compromised experimental treatment from the ones that represent true biological outliers.

To overcome this problem, academic and commercial laboratories have started to employ exogenous spike-in sequences that can mimic endogenous target RNA populations and, thus, allow the monitoring of experimental inefficiencies. The most commonly used spike-in for small RNA analysis is cel-miR-39-3p from the C. elegans worm. However, the use of a single spike-in e.g. cel-miR-39-3p on its own has several shortcomings. Firstly, a single spike-in does not represent well the considerable primary sequence heterogeneity of the entire small RNA complement. Since primary sequence specific effects such as RNA secondary structure, free energy and GC content can have a severe influence on the extraction and detection efficiency, a single species of spike-in is insufficient to reflect this in a faithful manner. Furthermore, a single spike-in cannot assess the bona fide preservation of relative abundances of different RNA levels. Linearity as the gold-standard for nucleic acid isolation and detection cannot be assessed from a single data point. Rather, in order to robustly assess the linearity of an experiment at least three distinct spike-ins, administered at distinct concentrations, would be required to calculate a Pearson coefficient.

To overcome the aforementioned caveats of using a single spike-in such as cel-miR-39-3p, the present inventors have designed and optimized a universal spike-in system of artificial small RNA molecules that broadly reflects endogenous miRNA behaviour during RNA extraction and detection. This concoction of artificial small RNA molecules is then added to biological samples such as clinical samples at the processing start and serves as an end-to-end quality control measure: only those biological samples such as clinical samples of which the analysis resulted in the recovery of the artificial small RNA molecules in their expected level, order, and linearity qualify are used for downstream analysis. Furthermore, the assessment of the cocktail of artificial small RNA molecules can function as bioinformatic normalization tool to perform batch effect removals on different experiments.

In addition, the present inventors have found that the artificial small RNA molecules can be used to identify undesired attachments to target RNA molecules. They have found that the exclusion of target RNA molecules with such attachments improves RNA data quality and, thus, target RNA analysis.

The artificial small RNA molecules are also designated as spike-ins.

### SUMMARY OF THE INVENTION

In a first aspect, the present invention relates to a composition comprising at least 3 RNA molecules, wherein the at least 3 RNA molecules are selected from a group consisting of RNA molecules having a nucleotide sequence according to SEQ ID NO: 1 to SEQ ID NO: 4, a fragment thereof, and a sequence having at least 80%, preferably at least 85%, more preferably at least 90%, and even more preferably at least 95%, e.g. 80, 81, 82, 83, 84, 85, 86, 87, 88, 89, 90, 91, 92, 93, 94, 95, 96, 97, 98, or 99%, sequence identity thereto.

In a second aspect, the present invention relates to a kit comprising the composition of the first aspect.

In a third aspect, the present invention relates to the use of the composition of the first aspect or the kit of the second aspect (as standard) for process control, sample examination, normalization, and/or data processing control. Preferably, the data processing control comprises/is raw data processing control.

In a fourth aspect, the present invention relates to a method for examining a sample comprising the step of:
evaluating a sample with respect to the at least 3 RNA molecules comprised in/from the composition of the first aspect.

In a fifth aspect, the present invention relates to a method for optimized processing of biological samples comprising the step of:
carrying out the method of the fourth aspect.

In a sixth aspect, the present invention relates to a method for optimized RNA preparation from or RNA analysis of biological samples comprising the step of:
carrying out the method of the fourth aspect.

In a seventh aspect, the present invention relates to a method for improving (RNA) data set quality comprising the step of:
carrying out the method of the fourth aspect.

In an eight aspect, the present invention relates to a method for improving (RNA) data set quality comprising the steps of:
(i) determining the sequence of RNA molecules in a sample,
(ii) determining 5′end and/or 3′end additions to the RNA molecules, which are not part of the RNA molecules in naturally occurring form, and
(iii) excluding RNA molecules having 5′end and/or 3′end additions from (RNA) data set analysis/removing RNA molecules having 5′end and/or 3′end additions from the (RNA) data set.

Preferably, the (RNA) data set analysis is raw (RNA) data analysis or the (RNA) data set is a raw (RNA) data set.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

As used herein, the term “about” indicates a certain variation from the quantitative value it precedes. In particular, the term “about” allows a ±5% variation from the quantitative value it precedes, unless otherwise indicated or inferred. The use of the term “about” also includes the specific quantitative value itself, unless explicitly stated otherwise. For example, the expression “about 80° C.” allows a variation of ±4° C., thus referring to range from 76° C. to 84° C.

The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90:5873-5877), with hmmalign (HMMER package) or with the CLUSTAL algorithm (Thompson, J. D., Higgins, D. G. & Gibson, T. J. (1994) Nucleic Acids Res. 22, 4673-80) or the CLUSTALW2 algorithm (Larkin M A, Blackshields G, Brown N P, Chenna R, McGettigan P A, McWilliam H, Valentin F, Wallace I M, Wilm A, Lopez R, Thompson J D, Gibson T J, Higgins D G. (2007). Clustal W and Clustal X version 2.0. Bioinformatics, 23, 2947-2948).

The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215:403-410. BLAST protein searches are performed with the BLASTP program available e.g. on the web site: http://blast.ncbi.nlm.nih.gov/Blast.cgi?PROGRAM=blastp&BLAST_PROGRAMS=blastp&PA GE_TYPE-BlastSearch&SHOW_DEFAULTS-on&LINK_LOC-blasthome

Preferred algorithm parameters used are the default parameters as they are set on the indicated web site:

Expect threshold=10, word size=3, max matches in a query range=0, matrix=BLOSUM62, gap costs=Existence: 11 Extension: 1, compositional adjustments=conditional compositional score matrix adjustment together with the database of non-redundant protein sequences (nr).

To obtain gapped alignments for comparative purposes, Gapped BLAST is utilized as described in Altschul et al. (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:I54-I62) or Markov random fields.

The term “nucleotide”, as used herein, refers to an organic molecule consisting of a nucleoside and a phosphate. In particular, a nucleotide is composed of three subunit molecules: a nucleobase, a five-carbon sugar (ribose or deoxyribose), and a phosphate group consisting of one to three phosphates. The four nucleobases in DNA are guanine, adenine, cytosine and thymine; in RNA, uracil is used in place of thymine. The nucleotide serves as monomeric unit of nucleic acid polymers, such as deoxyribonucleotide acid (DNA) or ribonucleotide acid (RNA). Thus, the nucleotide is a molecular building-block of DNA and RNA.

The terms “nucleotide sequence” or “polynucleotide” are interchangeably used herein and refer to single-stranded and double-stranded polymers of nucleotide monomers, including without limitation, 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+, and the like. A nucleotide sequence or polynucleotide may be composed entirely of deoxyribonucleotides, entirely of ribonucleotides, or chimeric mixtures thereof and may include nucleotide analogs. The nucleotide monomer units may comprise any of the nucleotides described herein, including, but not limited to, nucleotides and/or nucleotide analogs.

The term “nucleic acid molecule”, as used herein, refers to a single-stranded and double-stranded polymer of nucleotide monomers, including without limitation, 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+, and the like. A nucleic acid molecule may be composed entirely of deoxyribonucleotides, entirely of ribonucleotides, or chimeric mixtures thereof and may include nucleotide analogs.

The term “RNA molecule”, as used herein, refers to a polymeric form of ribonucleotides of any length. Like DNA, RNA is assembled as a chain of nucleotides, but unlike DNA, RNA is found in nature as a single strand folded onto itself, rather than a paired double strand.

Cellular organisms use messenger (mRNA) to convey genetic information (using the nitrogenous bases of guanine, uracil, adenine, and cytosine, denoted by the letters G, U, A, and C) that directs synthesis of specific proteins. Some RNA molecules play an active role within cells by catalysing biological reactions, controlling gene expression or sensing and communicating responses to cellular signals. One of these active processes is protein synthesis, a universal function in which RNA molecules direct the synthesis of proteins on ribosomes. This process uses transfer RNA

(IRNA) molecules to deliver amino acids to the ribosome, where ribosomal RNA (rRNA) then links amino acids together to form coded proteins.

In the context of the present invention, the RNA molecule is an artificial RNA molecule. Thus, it does not exist in nature. It is exogenous to a cell. It is synthetically designed/produced. The RNA molecule is comprised in a composition which can be used as universal spike-in system or cocktail.

The present inventors have designed and optimized RNA molecules that broadly reflect endogenous miRNA behaviour during RNA extraction and detection. Specifically, the present inventors generated 20 random sequences of 21 nucleotides in length (reflecting the typical length of miRNAs). Next, they evaluated their molecular characteristics e.g. melting temperature (Tm ° C.) and GC %. Afterwards, they selected a short list of 10 artificial sequences for wet lab validation on the basis of several criteria. Specifically, they wanted to minimize primer dimers and select RNAs of relatively weak secondary structure. They chose RNA molecules as spike-ins that range from 38.1% to 61.9% GC content which encompasses the majority of endogenous miRNA GC contents. Finally, the present inventors selected 4 artificial RNA molecules having a nucleotide sequence according to SEQ ID NO: 1 to SEQ ID NO: 4 as components of a composition which can be used as universal spike-in system or cocktail. At least 3 of these 4 RNA molecules have to be present in the composition in order to efficiently monitor experimental inefficiencies. For example, in order to robustly assess the linearity of an experiment, at least three distinct RNA molecules (spike-ins), administered at distinct concentrations, are required for statistical analysis, e.g. to calculate a Pearson coefficient.

Said RNA molecules are characterized by
a secondary structure which minimizes primer dimer formation,
a G/C content between 38.1 and 61.9% and, thus, encompass the majority of endogenous small RNA such as miRNA G/C contents, and
a 5′ phosphate group to mirror endogenous mature small RNA such as miRNAs.

The term “small RNA molecule”, as described herein, refers to a polymeric RNA molecule that is less than 200 ribonucleotides, preferably less than 50 ribonucleotides, in length. Specifically, small RNA molecules have a length of between 10 and <200 ribonucleotides. More specifically, small RNA molecules have a length of between 10 and <50 ribonucleotides. Small RNA molecules are usually non-coding RNA molecules. The RNA molecules having a nucleotide sequence according to SEQ ID NO: 1 to SEQ ID NO: 4 are small RNA molecules.

The term “composition”, as used herein, refers to a composition comprising at least 3 RNA molecules, wherein the at least 3 RNA molecules are selected from a group consisting of RNA molecules having a nucleotide sequence according to SEQ ID NO: 1 to SEQ ID NO: 4. The RNA molecules comprised in the composition are artificial, exogenous small RNA molecules. They do not exist in nature. In addition, they broadly reflect endogenous miRNA behaviour during RNA extraction and detection.

In one embodiment, the at least 3 RNA molecules comprised in the composition have a characteristic distribution, specifically with respect to their amounts. Particularly, the at least 3 RNA molecules are comprised in the composition in different amounts. More particularly, the at least 3 RNA molecules are comprised in the composition in a gradient of defined amounts. Specifically, the at least 3 RNA molecules are titrated to a linear range of different amounts.
Said composition can be used as spike-in cocktail or can be considered as spike-in cocktail. The spike-in cocktail is an universal spike-in cocktail that is agnostic of the downstream detection method.

The term “spiking-in”, as used herein, means adding (spiking) a known amount/quantity of an RNA molecule to a sample. The sample may comprise a biological material. The sample may also be a processed sample based on/derived from a biological material. Then a method is run to measure the response (recovery) of the spiked sample. The method can be run at any process step to determine the quantity or quality of the processing of the sample and/or analysis of the sample. For example, the method can be run after biological material lysis, RNA extraction, RNA or DNA (DNA derived from the RNA) amplification, and/or DNA (DNA derived from the RNA) sequencing.

The term “spike-in cocktail”, as used herein, refers to a composition comprising RNA molecules (also called RNA spike-ins) of known sequence and amount/quantity. The RNA molecules comprised in the composition are artificial, exogenous small RNA molecules. They do not exist in nature. In addition, they broadly reflect endogenous miRNA behaviour during RNA extraction and detection.

The spike-in cocktail of the present invention is a composition comprising at least 3 RNA molecules, wherein the at least 3 RNA molecules are selected from a group consisting of RNA molecules having a nucleotide sequence according to SEQ ID NO: 1 to SEQ ID NO: 4.
Said RNA molecules are added to a sample, such as biological sample or processed biological sample, to access performance of molecular biological experiments like nucleic acid quantification experiments, e.g. qPCR, next generation sequence (NGS), and/or microarray experiments.
As mentioned above, the at least 3 RNA molecules comprised in the composition have a characteristic distribution, specifically with respect to their amounts. Particularly, the at least 3 RNA molecules are comprised in the composition in different amounts. More particularly, the at least 3 RNA molecules are comprised in the composition in a gradient of defined amounts. Thus, any RNA molecule comprised in the composition is present in a specific and known amount which differs from the amount of the other RNA molecules. Specifically, the at least 3 RNA molecules are titrated to a linear range of different amounts.

In the method of the present invention relating to the examination of a sample, a sample is evaluated with respect to the at least 3 RNA molecules comprised in/from the composition described herein, wherein the at least 3 RNA molecules are selected from a group consisting of RNA molecules having a nucleotide sequence according to SEQ ID NO: 1 to SEQ ID NO: 4. The at least 3 RNA molecules are part of the sample. They have been added to the sample.

In this method, it is evaluated whether the at least 3 RNA molecules (during/after sample processing and analysis) still have this characteristic distribution. Specifically, it is evaluated, whether the at least 3 RNA molecules are present in their expected level, expected order, and/or expected linearity. In this way, the quality or quantity of the processing of the sample and/or analysis of the sample can be controlled.

In this respect, it will be appreciated that the term “RNA molecule” may refer to the RNA molecule itself as well as to surrogates thereof, for example, amplification products (e.g. cDNA derived therefrom).

The term “expected level”, as used herein, means the level which is expected for the specific RNA molecule amount added to the sample or comprised in the composition in subsequent sample processing and/or analysis. As mentioned above, the RNA molecule is added to the sample or comprised in the composition in a specific amount. The amount of the RNA molecule may correspond to/correlate with any expected level of the RNA molecule which can be measured during subsequent sample processing and/or analysis. Specifically, the amount of the RNA molecule may correspond to/correlate with specific read counts (reads per million (RPM)) in a next generation sequencing assay and/or the amount of the RNA molecule may correspond to/correlate with a specific cycle threshold (Ct) in a real-time PCR experiment. In particular, the cycle threshold (Ct) is the number of cycles in a real-time PCR that are required to exceed a previously defined threshold in the measurement signal (e.g. a fluorescence signal) of the amplified DNA. The more DNA (RNA) was already present in a sample solution before PCR, the fewer amplification cycles are required to reach the corresponding threshold value.

For example, the RNA molecule having a nucleotide sequence according to SEQ ID NO: 1 added to the sample or comprised in the composition of the present invention in an amount of about 3400 amol will result in a predicted Ct value of about 26 (Ct mean measured of about 24.3), the RNA molecule having a nucleotide sequence according to SEQ ID NO: 2 added to the sample or comprised in the composition of the present invention in an amount of about 725 amol will result in a predicted Ct value of about 29.3 (Ct mean measured of about 27.1), the RNA molecule having a nucleotide sequence according to SEQ ID NO: 3 added to the sample or comprised in the composition of the present invention in an amount of about 20 amol will result in a predicted Ct value of about 35.9 (Ct mean measured of about 32), and/or the RNA molecule having a nucleotide sequence according to SEQ ID NO: 4 added to the sample or comprised in the composition of the present invention in an amount of about 7 amol will result in a predicted Ct value of about 39.2 (Ct mean measured of about 33.6).
For example, the RNA molecule having a nucleotide sequence according to SEQ ID NO: 1 added to the sample or comprised in the composition of the present invention in an amount of about 3400 amol will result in a log2 RPM of about 11.972 or in read counts of about 30628, the RNA molecule having a nucleotide sequence according to SEQ ID NO: 2 added to the sample or comprised in the composition of the present invention in an amount of about 725 amol will result in a log2 RPM of about 10.926 or in read counts of about 5554, the RNA molecule having a nucleotide sequence according to SEQ ID NO: 3 added to the sample or comprised in the composition of the present invention in an amount of about 20 amol will result in a log2 PRM of about 6.559 or in read counts of about 491, and/or the RNA molecule having a nucleotide sequence according to SEQ ID NO: 4 added to the sample or comprised in the composition of the present invention in an amount of about 7 amol will result in a log2 RPM of about 4.061 or in read counts of about 94.

The term “level”, as used herein, refers to an amount (measured for example in grams, mole, or ion counts) or concentration (e.g. absolute or relative concentration, e.g. reads per million (RPM) or NGS counts) of RNA molecules comprised in a composition or sample. The term “level”, as used herein, also comprises scaled, normalized, or scaled and normalized amounts or values (e.g. RPM). In particular, the level of the RNA molecules is determined by sequencing, preferably next generation sequencing (e.g. ABI SOLID, Illumina Genome Analyzer, Roche 454 GS FL, BGISEQ), nucleic acid hybridization (e.g. microarray or beads), nucleic acid amplification (e.g. PCR, RT-PCR, qRT-PCR, or high-throughput RT-PCR), polymerase extension, mass spectrometry, flow cytometry (e.g. LUMINEX), or any combination thereof. Specifically, the level of the RNA molecules is the expression level of said RNA molecules.

The term “expected order”, as used herein, means the order/rank which is expected for the specific RNA molecules added to the sample or comprised in the composition in subsequent sample processing and/or analysis. As mentioned above, the RNA molecules added to the sample or comprised in the composition differ in their respective amounts. Thus, in case 3 RNA molecules are added to the sample or comprised in the composition, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules. In case 4 RNA molecules are added to the sample or comprised in the composition, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules, and the fourth RNA molecule (e.g. the RNA molecule having a nucleotide sequence according to SEQ ID NO: 4) is present in a smaller amount than the first, second, and third RNA molecules.

This expected order/rank, representing the initial state/condition, must be recovered during sample processing and/or analysis. In this respect, it should be noted that the amount of the RNA molecule comprised in a specific order/rank in the sample or composition may correspond to/correlate with any expected level of the RNA molecule which can be measured during subsequent sample processing and/or analysis. For example, the amount of the RNA molecule may correspond to/correlate with specific read counts in a next generation sequencing assay and/or the amount of the RNA molecule may correspond to/correlate with a specific cycle threshold (Ct) in a real-time PCR experiment.

The term “expected linearity”, as used herein, means the linearity which is expected for the specific RNA molecule added to the sample or comprised in the composition in subsequent sample processing and/or analysis.

This expected linearity, representing the initial state/condition, must be recovered during sample processing and/or analysis.

Only those samples of which the analysis resulted in the recovery of the RNA molecules or surrogates thereof in their expected level, order, and/or linearity are used for downstream analysis and/or further processing.

To determine, whether the RNA molecules are present in their expected order and/or linearity, statistical analysis are performed with data (e.g. Ct values or RPM) relating to said RNA molecules. Said statistical analysis include, but are not limited to, Spearman (rank) correlation analysis and/or Pearson correlation analysis.

For example, in order to determine, whether the RNA molecules are present in their expected order, the Spearman's rank correlation coefficient (Spearman's ρ) is preferably determined. In addition, in order to determine, whether the RNA molecules are present in their expected linearity, the Pearson's correlation coefficient (Pearson's r) is preferably determined.

The term “Spearman's rank correlation coefficient or Spearman's ρ”, as used herein, is a non-parametric measure of rank correlation (statistical dependence between the rankings of two variables). It assesses how well the relationship between two variables can be described using a monotonic function.

The Spearman correlation between two variables is equal to the Pearson correlation between the rank values of those two variables; while Pearson's correlation assesses linear relationships, Spearman's correlation assesses monotonic relationships (whether linear or not). If there are no repeated data values, a perfect Spearman correlation of +1 or −1 occurs when each of the variables is a perfect monotone function of the other.
Intuitively, the Spearman correlation between two variables will be high when observations have a similar (or identical for a correlation of 1) rank (i.e. relative position label of the observations within the variable: 1st, 2nd, 3rd, etc.) between the two variables, and low when observations have a dissimilar (or fully opposed for a correlation of −1) rank between the two variables.
In the context of the present invention, the RNA molecules are present in their expected order when the Spearman's rank correlation coefficient (Spearman's ρ) is ≥0.95. In this case, the sample is further processed/analysed. Thus, a Spearman's rank correlation coefficient (Spearman's ρ) of <0.95 leads to the discard of the sample. In other words, such a sample is not further processed/analyzed.

The term “Pearson correlation coefficient or Pearson's r”, as used herein, is a measure of linear correlation between two sets of data. It is the ratio between the covariance of two variables and the product of their standard deviations. Thus, it is essentially a normalized measurement of the covariance, such that the result always has a value between −1 and 1. As with covariance itself, the measure can only reflect a linear correlation of variables, and ignores many other types of relationship or correlation.

In the context of the present invention, the RNA molecules are present in their expected linearity when the Pearson's correlation coefficient (Pearson's r) of ≥0.66. In this case, the sample is further processed/analysed. Thus, a Pearson's correlation coefficient (Pearson's r) of <0.66 leads to the discard of the sample. In other words, such a sample is not further processed/analyzed.

Furthermore, the assessment of the cocktail of artificial small RNA molecules can function as bioinformatic normalization tool to perform batch effect removals on different experiments. The term “normalization”, as used herein, refers to a technique which is required to compare RNA levels across different samples. For example, normalization of high-throughput small RNA sequencing data is required to compare small RNA levels across different samples. Commonly used relative normalization approaches can cause erroneous conclusions due to fluctuating small RNA populations between tissues or bodily fluids. The present inventors developed a composition of RNA molecules (also called RNA spike-ins) that enable absolute normalization of small RNA data across independent experiments. Data from small RNA sequencing experiments are typically normalized and reported in relative terms such as reads per million genome-matching reads (RPMs). Relative normalization works well if it is assumed that the small RNA sub-populations have equal proportions across the different tissue or bodily fluid types being profiled. However, this assumption is often invalid because small RNA populations are frequently dynamic across different tissue or bodily fluid types and in various mutant backgrounds. Therefore, the standard practice of comparing relatively normalized small RNA-sequencing values can produce misleading results. In contrast, absolute normalization of small RNA-sequencing data should enable accurate comparisons of small RNA levels in different cell types, mutant tissues or disease states on a genome-wide scale. The present inventors have designed RNA molecules (also called RNA spike-ins) which can be used for robust absolute normalization, e.g. of small RNA sequencing data across independent experiments.

Said RNA molecules are characterized by
a secondary structure which minimizes primer dimer formation,
a G/C content between 38.1 and 61.9% and, thus, encompass the majority of endogenous small RNA such as miRNA G/C contents, and
a 5′ phosphate group to mirror endogenous mature small RNA such as miRNAs.

The term “quality”, as used herein, relative to a sample (comprising a biological material) refers to the level of degradation of components in the sample relative to when the components were comprised in a biological system, such as a cell. For example, assessing the quality of RNA and/or DNA can comprise assessing the level of partial degradation of RNA and/or DNA polymers. In some embodiments, assessing quality comprises assessing partial degradation of RNA and/or DNA from a spike-in standard, e.g. by assessing the presence of fragments (shorter molecules) of spike-in standard with relation to the full-length spike-in standard.

The term “quantity”, as used herein, relative to a sample (comprising a biological material) refers to the level of a component present in the sample relative to when the components were comprised in a biological system, such as a cell. In some embodiments, assessing the quantity of RNA and/or DNA can comprise assessing the level of degradation or loss of RNA and/or DNA polymers in a sample.

The term “sample”, as used herein, refers to a mixture of a biological material with the composition of the present invention. Alternatively, the sample is based on/derived from a mixture of a biological material with the composition of the present invention. In this case, the sample is often a processed sample. The processed sample is obtained by mixing a biological material with the composition of the present invention and further processing the mixture obtained thereby. For example, the processed sample is a lysed sample, an extracted sample, an amplified sample, a sequenced sample, or a library prepped sample.

The term “biological material”, as used herein, refers to any material having a biological origin. The biological material is preferably a tissue material or a body fluid material.

The term “body fluid material”, as used herein, refers to any liquid material derived from the body of an individual.

Said body fluid material may be urine, blood, sputum, breast milk, cerebrospinal fluid (CSF), cerumen (earwax), gastric juice, mucus, lymph, endolymph fluid, perilymph fluid, peritoneal fluid, pleural fluid, saliva, sebum (skin oil), semen, sweat, tears, cheek swab, vaginal secretion, liquid biopsy, or vomit sample including components or fractions thereof. The term “body fluid material” also encompasses body fluid fractions, e.g. blood fractions such as blood cells, serum or plasma, wherein blood cells represent the cellular fraction of blood and serum as well plasma represent the acellular fraction of blood.

The term “blood material”, as used herein, encompasses whole blood or a blood fraction. Preferably, the blood fraction is selected from the group consisting of a blood cell fraction, plasma, and serum. For example, the blood cell fraction encompasses erythrocytes, leukocytes, and/or thrombocytes. More preferably, the blood cell fraction is a fraction of leukocytes or a mixture of erythrocytes, leukocytes, and thrombocytes.

Said blood material may be provided by removing blood from an individual, but may also be provided by using a previously isolated material. For example, a blood material may be taken from an individual by conventional blood collection techniques.
The whole blood material may be collected by means of a blood collection tube. It is, for example, collected in a PAXgene Blood RNA tube, in a Tempus Blood RNA tube, in an EDTA-tube, in a Na-citrate tube, Heparin-tube, or in an ACD-tube (Acid citrate dextrose).
The blood material, in particular whole blood material, as used herein, may also be collected by means of a bloodspot technique, e.g. using a Mitra Microsampling Device. This technique requires smaller sample volumes, typically 45-60 μl for humans or less. For example, the whole blood may be extracted from the individual via a finger prick with a needle or lancet. Thus, the whole blood material may have the form of a blood drop. Said blood drop is then placed on an absorbent probe, e.g. a hydrophilic polymeric material such as cellulose, which is capable of absorbing the whole blood. Once sampling is complete, the blood spot is dried in air before transferring or mailing to labs for processing. Because the blood is dried, it is not considered hazardous. Thus, no special precautions need be taken in handling or shipping. Once at the analysis site, the desired components, e.g. the RNAs, are extracted from the dried blood spots into a supernatant which is then further analyzed. In this way, the level of the RNAs is determined.

The term “tissue material” as used herein, refers to any tissue material derived from the body of an individual. Said material may be tumor/cancerous tissue material or healthy tissue material of any organ of an individual. For example, the tissue material may be material of the lungs, kidney, liver, brain, colon, breast, stomach, uterus, ovarian, pancreas, or prostate. It can be removed from the individual by conventional biopsy techniques.

The term “target RNA”, as used herein, refers to an endogenous ribonucleotide sequence comprised in a biological material that is sought to be detected. The target RNA may be obtained from any source and may comprise any number of different compositional components. For example, the target RNA is isolated from organisms, tissues, cells, or bodily fluids such as blood. Preferably, the target RNA encompasses non-coding RNA. In particular, the target RNA is a microRNA (miRNA) or a miRNA isoform (an isomiR) and may comprise variants, analogs, and mimics. The target RNA may, in some cases, also be designated as target RNA molecule/molecules.

Further, it will be appreciated that the term “target RNA” may refer to the target molecule itself as well as to surrogates thereof, for example, amplification products (e.g. cDNA derived therefrom) and native sequences. In certain embodiments, the target RNA is a miRNA or miRNA isoform (an isomiR) molecule. In certain embodiments, the target RNA is a mature small RNA molecule, in particular a non-coding small RNA molecule (i.e. having a length of <200 ribonucleotides, e.g. between 10 and <200 ribonucleotides). The target RNA described herein may be derived from any number of sources, including without limitation, humans and animals. These sources may include, but are not limited to, whole blood, a tissue biopsy, lymph, bone marrow, amniotic fluid, hair, skin, semen, biowarfare agents, anal secretions, vaginal secretions, perspiration, saliva, or buccal swabs. However, various environmental samples (for example, agricultural, water, and soil), research samples generally, purified samples generally, cultured cells and lysed cells may also be used as samples. It will be appreciated that target RNAs may be isolated from samples using any of a variety of procedures known in the art, for example, the Applied Biosystems ABI Prism® 6100 Nucleic Acid PrepStation (Life Technologies, Foster City, CA) and the ABI Prism® 6700 Automated Nucleic Acid Workstation (Life Technologies, Foster City, CA), Ambion® mirVana™ RNA isolation kit (Life Technologies, Austin, TX), PAXgene Blood RNA Kit (Qiagen, Hilden, Germany) and the like.
The target RNA molecule having an endogenous origin in the biological material differs from the exogenous artificial RNA molecule also described herein. While the exogenous artificial RNA molecule is added, as part of a composition such as spike-in cocktail, to a biological material, the target RNA molecule is part of said biological material.

The term “miRNA” (the designation “microRNA” is also possible), as used herein, refers to a single-stranded RNA molecule. The miRNA may be a molecule of 10 to 50 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, not including optionally labels and/or elongated sequences (e.g. biotin stretches).

The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNA is initially transcribed as a longer precursor molecule (>1000 nucleotides long) called a primary miRNA transcript (pri-miRNA). Pri-miRNAs have hairpin structures that are processed by the Drosha enzyme (as part of the microprocessor complex). After Drosha processing, the pri-miRNAs are only 60-100 nucleotides long, and are called precursor miRNAs (pre-miRNAs). At this point, the pre-miRNA is exported to the cytoplasm, where it encounters the Dicer enzyme. Dicer cuts the miRNA in two, resulting in duplexed miRNA strands. Traditionally, only one of these miRNA arms was considered important in gene regulation: the arm that is destined to be loaded into the RNA-induced silencing complex (RISC), and occurs at a higher concentration in the cell. This is often called the “guide” strand and is designated as miR. The other arm is called the “minor miRNA” or “passenger miRNA”, and is often designated as miR*. It was thought that passenger miRNAs were completely degraded, but deep sequencing studies have found that some minor miRNAs persist and in fact have a functional role in gene regulation. Due to these developments, the naming convention has shifted. Instead of the miR/miR* name scheme, a miR-5p/miR-3p nomenclature has been adopted. By the new system, the 5′ arm of the miRNA is always designated miR-5p and the 3′ arm is miR-3p. The present nomenclature is as follows: The prefix “miR” is followed by a dash and a number, the latter often indicating order of naming. For example, hsa-miR-16 was named and likely discovered prior to hsa-miR-342. A capitalized “miR-” refers to the mature forms of the miRNA (e.g. hsa-miR-16-5p and hsa-miR-16-3p), while the uncapitalized “mir-” refers to the pre-miRNA and the pri-miRNA (e.g. hsa-mir-16), and “MIR” refers to the gene that encodes them. However, as this is a recent change, literature will often refer to the original miR/miR* names. After processing, the duplexed miRNA strands are loaded onto an Argonaute (AGO) protein to form a precursor to the RISC. The complex causes the duplex to unwind, and the passenger RNA strand is discarded, leaving behind a mature RISC carrying the mature, single stranded miRNA. The miRNA remains part of the RISC as it silences the expression of its target genes. While this is the canonical pathway for miRNA biogenesis, a variety of others have been discovered. These include Drosha-independent pathways (such as the mirtron pathway, snoRNA-derived pathway, and shRNA-derived pathway) and Dicer-independent pathways (such as one that relies on AGO for cleavage, and another which is dependent on tRNaseZ).

The term “miRBase”, as used herein, refers to a well-established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download. In October 2018, miRbase version 22.1 was released. This is the current version.

The term “isomiR” (or “miRNA isoform”), as used herein, refers to a miRNA that varies slightly in sequence, which results from variations in the cleavage site during miRNA biogenesis. In particular, imprecise cleavage of Drosha and Dicer or the turnover of miRNAs can result in miRNAs that are heterogeneous in length and/or sequence. IsomiRs (miRNA isoforms) can be divided into three main categories: 3′ isomiRs (trimmed or addition of one or more nucleotides at the 3′ position), 5′isomiRs (trimmed or addition of one or more nucleotides at the 5′ position), and polymorphic isomiRs (some nucleotides within the sequence are different from the wild type mature miRNA sequence). It could be envisioned that the increased expression of miRNA variants, or individual isomiRs, lead to the loss or weakening of the function of the corresponding wild-type mature miRNA or result in the regulation of a different transcriptome. Recent studies suggest that isomiRs probably play vital roles in a variety of cancers, tissues, and cell types. The detection of miRNAs as well as isomiRs is, thus, absolutely required to accurately reflect the underlying biological situation and to make the right diagnostic and treatment decisions.

RNAs may be isolated from samples using any of a variety of procedures known in the art, for example, the Applied Biosystems ABI Prism® 6100 Nucleic Acid PrepStation (Life Technologies, Foster City, CA) and the ABI Prism® 6700 Automated Nucleic Acid Workstation (Life Technologies, Foster City, CA), Ambion® mirVana™ RNA isolation kit (Life Technologies, Austin, TX), PAXgene Blood RNA Kit (Qiagen, Hilden, Germany) and the like.

RNA molecule detection requires the presence of the RNA molecules in higher amounts. Due to the process of reverse transcription, cDNA molecules are produced from the RNA molecules. In various embodiments, these cDNA molecules are amplified. RNA molecules can also be amplified directly. The term “amplifying”, as used herein, refers to any means by which at least a part of a nucleic acid molecule described herein is reproduced, typically in a template-dependent manner, including without limitation, a broad range of techniques for amplifying nucleic acid sequences, either linearly or exponentially. Any of several methods can be used to amplify the nucleic acid molecule. Any in vitro means for multiplying the copies of a target sequence of nucleic acid can be utilized. These include linear, logarithmic, or other amplification methods.

Examples of amplification techniques that can be used include, but are not limited to, PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), hat start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid-based sequence amplification (NABSA).

In various embodiments, the DNA molecules derived from the RNA molecules are sequenced. The term “sequencing”, as used herein, includes any method of determining the sequence of a nucleic acid molecule. Such methods include Maxam-Gilbert sequencing, Chain-termination methods, Shot gun sequencing, PCR sequencing, Bridge PCR, massively parallel signature sequencing (MPSS), Polony sequencing, pyrosequencing, Illumina (Solexa) sequencing, SOLiD sequencing, Ion semiconductor sequencing, DNA nanoball sequencing, Heliscope single molecule sequencing, Single molecule real time (SMRT) sequencing, Nanopore DNA sequencing, sequencing by hybridization, sequencing with mass spectrometry, microfluidic Sanger sequencing, microscopy-based techniques, RNAP sequencing, (in vitro virus) high-throughput sequencing (HTS).

The term “next generation sequencing (NGS)” as used herein, refers to a new method for sequencing nucleotide sequences at high speed and at low cost. Next-generation sequencing (NGS) is, thus, a high-throughput methodology that enables rapid sequencing of the base pairs in DNA or RNA samples. Supporting a broad range of applications, including gene expression profiling, chromosome counting, detection of epigenetic changes, and molecular analysis, NGS is driving discovery and enabling the future of personalized medicine. NGS is also known as second generation sequencing (SGS) or massively parallel sequencing (MPS).

In the context of the present invention, the term “kit of parts (in short: kit)” is understood to be any combination of at least some of the components identified herein, which are combined, coexisting spatially, to a functional unit, and which can contain further components.

The term “data processing control”, as used herein, refers to an early step during data analysis where parts of the data are removed or reduced in size to improve aspects of later analysis, such as improving the quality of data and improving the reliability of findings during analysis. Preferably, the data processing control is raw data processing control.

The term “raw data”, as used herein, refers to data collected during experimentation. These data are not (yet) further processed. Raw data can be quantitative (numbers) and/or qualitative (descriptions). Raw data sizes (generally large), the file types, and structures depend on the technology that is used to produce said data. Raw data need to be processed using dedicated software before obtaining data that can finally be analysed, e.g. mapped to the biological situation/biology that is measured, or to find statistical differences between groups of biological samples such as patients with different medical conditions.

In the next generation sequencing process control, quality control and pre-processing of data are important for data analysis because raw data produced after sequencing must be processed so that the results should not have false positive and false negative results. Pre-processing of data not only evaluates each analysis step but also it reduces the amount of low-quality sequence reads. Removal of such low-quality reads decrease time and cost of computation analysis and also reliable and high-quality results are obtained.

In any downstream analysis of NGS data, false-positive and false-negative results are produced due to:
experimental factors: like sample contamination or PCR errors,
sequencing factors: these include quality of sequencing and data contamination caused due to index hopping while splitting data, and/or
parameters of analysis software factors: this includes alignment software or precise type of parameter adjustment of downstream personalized analysis.
   The raw sequences generated after sequencing not only contain the sequences of interest, e.g. of target RNA molecules, but they also have sequence biases (for instance through systematic effects like Poisson sampling) and complex artefacts which are generated due to sequencing and experimental steps. These sequence biases and artefacts affect and interfere with precise read alignments which influence the genotyping and variant calling. Therefore, in order to increase the reliability and quality of downstream analysis and reduce the amount of required computational resources, the pre-processing of raw sequence reads is a necessity.

The present inventors found that 5′end and/or 3′end additions to (target) RNA molecule, e.g. produced or occurring during sequencing processes, e.g. next generation sequencing processes, are misleading and falsify the raw data results. Specifically, adaptor contaminated sequence reads need to be eliminated/excluded. The present inventors found that by elimination/exclusion of adaptor contaminated sequence reads the quality of raw data can be improved.

The term “5′end additions (also designated as prefixes)”, as used herein, refers to nucleotides attached to the 5′end of the RNA molecule or molecule derived therefrom such as cDNA molecule. They are particularly produced/are the remnants of 5′ sequencing adapters or other RNAs present in the same physical mixture and that have become (fully or in part) fused during the technical process of sequencing. In the case of sequencing adapters, an intended fusion may not have been followed by an intended cleavage to remove the adapter after sequencing was complete.

The term “3′end additions (also designated as suffixes)”, as used herein, refers to nucleotides attached to the 3′end of the RNA molecule or molecule derived therefrom such as cDNA molecule. They are particularly produced/are the remnants of other RNAs present in the same physical mixture and that have become (fully or in part) fused during the technical process of sequencing. In this respect, it will be appreciated that the term “RNA molecule” may refer to the RNA molecule itself as well as to surrogates thereof, for example, amplification products (e.g. cDNA derived therefrom).

Specifically, during next generation sequencing (NGS) parts of other RNAs or adapter sequences are found attached to (target) RNA molecules on one end or on both ends (5′end and/or 3′end). These parts are here called affixes, which include both prefixes (5′) and suffixes (3′).

Preferably, the 5′end and/or 3′end additions have a length of between 5 and 30 nucleotides, more preferably between 5 and 20 nucleotides, and even more preferably between 7 and 15 nucleotides, e.g. 5, 6, 7, 8, 9, 10, 11, 12, 13, 14, 15, 16, 17, 18, 19, 20, 21, 22, 23, 24, 25, 26, 27, 28, 29, or 30 nucleotides.

Specifically, the 5′end and/or 3′ end additions take place/occur during:
ligation of 5′adapters and/or 3′adapters to (denatured) (target) RNA molecules, e.g. using/with a double stranded RNA ligase such as T4 RNA ligase 2 (Rnl2) or a Kod1 ligase,
reverse transcription of (said) (target) RNA molecules (having 5′adapters and/or 3′adapters ligated thereon, also designated as ligation products) into cDNA molecules, e.g. using/with a reverse transcriptase (RT) such as Maxima H-RT or Tth polymerase,
amplification of (said) cDNA molecules, e.g. via polymerase chain reaction (PCR), and/or
sequencing such as next generation sequencing of (said) cDNA molecules.
   The PCR may be real-time PCR (quantitative PCR or qPCR), such as TaqMan qPCR, multiplex PCR, nested PCR, high fidelity PR, fast PCR, hot start PCR, or GC-rich PCR.

More specifically, the 5′end and/or 3′ end additions take place/occur during library preparation process for next generation sequencing or during the next generation sequencing process. Even more specifically,

the next generation sequencing process preferably encompasses:
ligation of 5′adapters and/or 3′adapters to (denatured) (target) RNA molecules, e.g. using/with a double stranded RNA ligase such as T4 RNA ligase 2 (Rnl2) or a Kod1 ligase,
reverse transcription of (said) (target) RNA molecules (having 5′adapters and/or 3′adapters ligated thereon, also designated as ligation products) into cDNA molecules, e.g. using/with a reverse transcriptase (RT) such as Maxima H-RT or Tth polymerase,
amplification of (said) cDNA molecules, e.g. via polymerase chain reaction (PCR), and/or
sequencing of (said) cDNA molecules.

The terms “5′end and/or 3′end additions” or “5′end and/or 3′end attachments” can interchangeably be used herein.

The term “adapter”, as used herein, refers to any non-biological RNA/DNA sequence that is intentionally added to the 5′end or 3′end of a biological (target) RNA/cDNA molecule (originating from the sample to be sequenced) as part of the design of a sequencing such as NGS method. This includes any free-floating adapter molecules that may fuse with biological RNA in an unintended manner. An adapter can also be a combination/fusion of multiple individual adapters and indexing RNAs. Indexing RNAs are used as UMI (unique molecular identifier) for assigning RNA molecules to their sample of origin during multiplexed sequencing, i.e. sequencing multiple samples at the same time. UMI sequences are short unspecific (random) sequences of a predefined length, e.g. 12 nucleotides, but can also vary in length and are incorporated in between other adapter sequences.

The term “RNA molecules in naturally occurring form”, as used herein, refers to RNA molecules in a form in which they occur in nature or in a natural environment, e.g. bodily fluid such as whole blood, or tissue. These RNA molecules may, however, be further processed, e.g. into cDNA molecules. In this case, they have a natural origin.

The term “RNA molecules in naturally occurring form”, as used herein, further refers to RNA molecules having an endogenous origin in a biological material/sample. Said RNA molecules are part of said biological material/sample and may occur in their natural functional form or may be a fragment of longer functional RNA sequences that have been degraded or processed either as part of a specific biological process or through unspecific physical/chemical forces over time.

### Embodiments of the Invention

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

Academic and commercial laboratories have started to employ exogenous spike-in sequences that can mimic endogenous target RNA populations and, thus, allow the monitoring of experimental inefficiencies. The most commonly used spike-in for small RNA analysis is cel-miR-39-3p from the C. elegans worm. However, the use of a single spike-in e.g. cel-miR-39-3p on its own has several shortcomings. Firstly, a single spike-in does not represent well the considerable primary sequence heterogeneity of the entire small RNA complement. Since primary sequence specific effects such as RNA secondary structure, free energy and GC content can have a severe influence on the extraction and detection efficiency a single species of spike-in is insufficient to reflect this in a faithful manner. Furthermore, a single spike-in cannot assess the bona fide preservation of relative abundances of different RNA levels. Linearity as the gold-standard for nucleic acid isolation and detection cannot be assessed from a single data point. Rather, in order to robustly assess the linearity of an experiment at least three distinct spike-ins, administered at distinct concentrations, would be required to calculate a Pearson coefficient.

To overcome the aforementioned caveats of using a single spike-in such as cel-miR-39-3p, the present inventors have designed and optimized a universal spike-in system of artificial small RNA molecules that broadly reflects endogenous miRNA behaviour during RNA extraction and detection. This concoction of artificial small RNA molecules is then added to a sample comprising a biological material such as clinical material at the processing start and serves as an end-to-end quality control measure: only those samples of which the analysis resulted in the recovery of the artificial small RNA molecules in their expected level, order, and linearity are used for downstream analysis. Furthermore, the assessment of the cocktail of artificial small RNA molecules can function as bioinformatic normalization tool to perform batch effect removals on different experiments.

In addition, the present inventors have found that the artificial small RNA molecules can be used to identify undesired attachments to target RNA molecules. They have found that the exclusion of target RNA molecules with such attachments improves RNA data quality and, thus, target RNA analysis.

The artificial small RNA molecules are also designated as spike-ins.

Thus, in a first aspect, the present invention relates to a composition comprising at least 3RNA molecules (e.g. 3 or 4 RNA molecules), wherein the at least 3 RNA molecules (e.g. 3 or 4RNA molecules) are selected from a group consisting of RNA molecules having a nucleotide sequence according to SEQ ID NO: 1 to SEQ ID NO: 4, a fragment thereof, and a sequence having at least 80%, preferably at least 85%, more preferably at least 90%, and even more preferably at least 95%, e.g. 80, 81, 82, 83, 84, 85, 86, 87, 88, 89, 90, 91, 92, 93, 94, 95, 96, 97, 98, or 99%, sequence identity thereto.

Specifically, the RNA molecule comprised in the composition
(i) has a nucleotide sequence according to SEQ ID NO: 1, 2, 3 or 4,
(ii) is a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) is a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

The RNA molecule with SEQ ID NO: 1 has the following nucleotide sequence: GAUAGAUACGCCAGUACCGCC,

the RNA molecule with SEQ ID NO: 2 has the following nucleotide sequence: AACGAAGCUCCACGAUGUAGG,
the RNA molecule with SEQ ID NO: 3 has the following nucleotide sequence: UGUACGGAAAUAUUGGCUACC, and
the RNA molecule with SEQ ID NO: 4 has the following nucleotide sequence: UUCAUACGUUGCCCAAUCCAG.

The RNA molecules comprised in the composition are artificial, exogenous small RNA molecules. They do not exist in nature. They are exogenous to a cell. In addition, they broadly reflect endogenous miRNA behaviour during RNA extraction and detection. Moreover, the RNA molecules comprised in the composition have
a secondary structure which minimizes primer dimer formation,
a G/C content between 38.1 and 61.9% and, thus, encompass the majority of endogenous small RNA such as miRNA G/C contents, and
a 5′ phosphate group to mirror endogenous mature small RNA such as miRNAs.

Preferably, the composition comprises 4 RNA molecules, wherein the 4 RNA molecules are selected from a group consisting of RNA molecules having a nucleotide sequence according to SEQ ID NO: 1 to SEQ ID NO: 4, a fragment thereof, and a sequence having at least 80%, preferably at least 85%, more preferably at least 90%, and even more preferably at least 95%, e.g. 80, 81, 82, 83, 84, 85, 86, 87, 88, 89, 90, 91, 92, 93, 94, 95, 96, 97, 98, or 99%, sequence identity thereto.

Thus, the composition can comprise RNA molecules having a nucleotide sequence according to
(i) SEQ ID NO: 1, SEQ ID NO: 2, and SEQ ID NO: 3,
(ii) SEQ ID NO: 2, SEQ ID NO: 3, and SEQ ID NO: 4,
(iii) SEQ ID NO: 1, SEQ ID NO: 3, and SEQ ID NO: 4,
(iv) SEQ ID NO: 1, SEQ ID NO: 2, and SEQ ID NO: 4, or
(v) SEQ ID NO: 1, SEQ ID NO: 2, SEQ ID NO: 3, and SEQ ID NO: 4.
   Fragments of the RNA molecules listed under (i) to (v), or sequences having at least 80%, preferably at least 85%, more preferably at least 90%, and even more preferably at least 95%, e.g. 80, 81, 82, 83, 84, 85, 86, 87, 88, 89, 90, 91, 92, 93, 94, 95, 96, 97, 98, or 99%, sequence identity to the RNA molecule listed under (i) to (v) are also encompassed.

It should be noted that the at least 3 RNA molecules, particularly 4 RNA molecules, comprised in the composition have a characteristic distribution. Especially, the at least 3 RNA molecules, particularly 4 RNA molecules, comprised in the composition have a characteristic distribution with respect to their amounts.

Specifically, the at least 3 RNA molecules, particularly 4 RNA molecules, are comprised in the composition in different amounts. For example, any arbitrary pair of 2 RNA molecules comprised in the composition have different amounts.

Thus, in case 3 RNA molecules are comprised in a composition, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules. In case 4 RNA molecules are comprised in a composition, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules, and the fourth RNA molecule (e.g. the RNA molecule having a nucleotide sequence according to SEQ ID NO: 4) is present in a smaller amount than the first, second, and third RNA molecules.

More specifically, the at least 3 RNA molecules, particularly 4 RNA molecules, are comprised in the composition in a gradient of defined amounts. Especially, the at least 3 RNA molecules, particularly 4 RNA molecules, are titrated to a linear range of different amounts.

In one embodiment, the at least 3 RNA molecules, particularly 4 RNA molecules, comprised in the composition are present in an amount of between 0.001 amol to 6000 amol, preferably in an amount of between 0.01 amol to 5000 amol, more preferably in an amount of between 0.1 amol to 4000 amol, and even more preferably in an amount of between 1 amol and 3500 amol, e.g. 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 3500, 4000, 4500, 5000, 5500, or 6000 amol.

In one example, the composition comprises at least 3 RNA molecules, wherein
the first RNA molecule is comprised in an amount of about 3400 amol, the second RNA molecule is comprised in an amount of about 725 amol, and the third RNA molecule is comprised in an amount of about 20 amol, or
the first RNA molecule is comprised in an amount of about 1360 amol, the second RNA molecule is comprised in an amount of about 290 amol, and the third RNA molecule is comprised in an amount of about 80 amol.
   Specifically, the first RNA molecule has a nucleotide sequence according to SEQ ID NO: 1, the second RNA molecule has a nucleotide sequence according to SEQ ID NO: 2, and the third RNA molecule has a nucleotide sequence according to SEQ ID NO: 3.

In one particular embodiment, the 4 RNA molecules comprised in the composition are present in an amount of between 0.001 amol to 6000 amol, preferably in an amount of between 0.01 amol to 5000 amol, more preferably in an amount of between 0.1 amol to 4000 amol, and even more preferably in an amount of between 1 amol and 3500 amol, e.g. 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 1500, 2000, 3000, 3500, 4000, 4500, 5000, 5500, or 6000 amol.

In one particular example, the composition comprises 4 RNA molecules, wherein
the first RNA molecule is comprised in an amount of about 3400 amol, the second RNA molecule is comprised in an amount of about 725 amol, the third RNA molecule is comprised in an amount of about 20 amol, and the fourth RNA molecule is comprised in an amount of about 7 amol, or
the first RNA molecule is comprised in an amount of about 1360 amol, the second RNA molecule is comprised in an amount of about 290 amol, the third RNA molecule is comprised in an amount of about 80 amol, and the fourth RNA molecule is comprised in an amount of about 27 amol. Specifically, the first RNA molecule has a nucleotide sequence according to SEQ ID NO: 1, the second RNA molecule has a nucleotide sequence according to SEQ ID NO: 2, the third RNA molecule has a nucleotide sequence according to SEQ ID NO: 3, and the fourth RNA molecule has a nucleotide sequence according to SEQ ID NO: 4.

Preferably, the composition is a solution. More preferably, the solution is an aqueous solution such as water like nuclease free water. Even more preferably, no other components (than the RNA molecules and water as solvent) are part of the composition.

As mentioned above, analysis of RNA expression profiles involves several intricate steps. (1) RNA, including small RNA, must be extracted from a biological source, e.g. blood or saliva, without distorting the original relative abundances of the RNA (linearity must be preserved). (2) The small RNA abundances must be reliably quantified. Since both steps can inconsistently introduce bias (e.g. through the presence of inhibitors of cDNA synthesis) it is crucial to standardize but also monitor both the efficiency of RNA extraction and detection.

The composition as described above is suitable as/is a spike-in cocktail. The spike-in cocktail is an universal spike-in cocktail that is agnostic of the downstream detection method. In addition, the composition is suitable as/is a standard for process control and/or normalization. For this purpose, the composition as described above is added to a sample comprising a biological material or to a processed sample based on/derived from a biological material.

In particular, the composition is universally applicable in any downstream processing of a biological material, sample comprising a biological material, sample comprising a material with biological origin, and/or sample based on/derived from a biological material.

Said biological material comprises target RNA molecules which are sought to be detected, e.g. in order to diagnose a disease such as cancer or a neurodegenerative disease like Parkinson's Disease (PD) or Alzheimer's Disease (AD).

The target RNA molecules have their endogenous origin in the biological material and differ from the exogenous artificial RNA molecules. While the exogenous artificial RNA molecules are added, as part of a composition such as spike-in cocktail, to a biological material, the target RNA molecules are part of said biological material.

The process control specifically encompasses: the monitoring/detection of experimental inefficiencies during target RNA processing and analysis, the monitoring of the efficiency of target RNA extraction and/or detection, the standardization of target RNA extraction and detection, the monitoring of target RNA quantification and/or detection.

The composition can be used as extraction control standard, sequencing control standard or library preparation control standard.

In addition, the composition can be used as normalization indicator (to compare to different samples with the composition). In particular, the composition can be used as normalization indicator (to compare to different samples with the composition) such that the measured expression values of spike-ins serve to control and correct for inefficiencies in extraction, library preparation or sequencing.

In a second aspect, the present invention relates to a kit comprising the composition of the first aspect.

The composition can also be designated as spike-in cocktail.

Preferably, the kit comprises means for determining the level of the at least 3 RNA molecules (e.g. 3 or 4 RNA molecules) comprised in the composition.

More preferably, the means are
polynucleotides (probes) for detecting the RNA molecules,
primers/primer pairs for binding the RNA molecules,
antibodies capable of binding hybrids of polynucleotide probes and said RNA molecules, and/or means for carrying out next generation sequencing (NGS).
   The polynucleotides (probes) may be part of a microarray/biochip or may be attached to beads of a beads-based multiplex system. The primers/primer pairs may be part of a RT-PCR system, a PCR-system, or a next generation sequencing system.
   Said means may further comprise a microarray, a RT-PCT system, a PCR-system, a flow cytometer, a Luminex system, and/or a next generation sequencing system.

The kit may comprise instructions on how to carry out the methods of the present invention (see fourth to sixth aspect). The kit is also useful for conducting the methods of the present invention (see fourth to sixth aspect).

In addition, the kit may comprise
a container, and/or
a data carrier.
   The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.
   Said data carrier may further comprise information with respect to the expected level, expected order/rank, and/or expected linearity of the RNA molecules comprised in the composition in subsequent RNA processing/analysis experiments.
   The data carrier may also comprise information or instructions on how to carry out the methods of the present invention (see fourth to sixth aspect).

The kit is preferably used in vitro/in in vitro.

In a third aspect, the present invention relates to the (in vitro) use of the composition of the first aspect or the kit of the second aspect (as standard) for process control, sample examination, sample analysis, normalization, and/or data processing control.

The composition can also be designated as spike-in cocktail.

Preferably, the data processing control comprises raw data processing control.

As mentioned above, analysis of RNA expression profiles involves several intricate steps. (1) RNA, including small RNA, must be extracted from a biological source, e.g. blood or saliva, without distorting the original relative abundances of the RNA (linearity must be preserved). (2) The small RNA abundances must be reliably quantified. Since both steps can inconsistently introduce bias (e.g. through the presence of inhibitors of cDNA synthesis) it is crucial to standardize but also monitor both the efficiency of RNA extraction and detection.

The composition of the first aspect is used (as standard) for process control, sample examination, sample analysis, normalization, and/or data processing control. Preferably, the data processing control comprises raw data processing control. For this purpose, the composition of the first aspect is added to a sample comprising a biological material or to a processed sample based on/derived from a biological material.

In particular, the composition is universally applicable in any downstream processing of a biological material, sample comprising a biological material, sample comprising a material with biological origin, and/or sample based on/derived from a biological material.

Said biological material comprises target RNA molecules which are sought to be detected, e.g. in order to diagnose a disease such as cancer or a neurodegenerative disease like Parkinson's Disease (PD) or Alzheimer's Disease (AD).

The target RNA molecules have an endogenous origin in the biological material and differ from the exogenous artificial RNA molecules. While the exogenous artificial RNA molecules are added, as part of a composition such as spike-in cocktail, to a biological material, the target RNA molecules are part of said biological material.

The process control specifically encompasses: the monitoring/detection of experimental inefficiencies during target RNA processing and analysis, the monitoring of the efficiency of target RNA extraction and/or detection, the standardization of target RNA extraction and detection, the monitoring of target RNA quantification and/or detection.

Preferably, the process control comprises quality control, quantity control, or end-to-end control. End-to-end control measure specifically means that only those samples of which the analysis resulted in the recovery of the artificial small RNA molecules in their expected level, order, and linearity are used for downstream analysis.

The composition of the first aspect can especially be used as extraction control standard, a sequencing control standard or a library preparation control standard.

In addition, the composition of the first aspect can especially be used as normalization indicator (to compare to different samples with the composition). This allows batch effect removals on different experiments and/or control for inefficiencies in the extraction, library preparation and/or sequencing process.

Process control, quality control and pre-processing of data are important for data analysis because data, specifically raw data, produced after sequencing such as next generation sequencing must be processed so that the results should not have false positive and false negative results. Pre-processing of data not only evaluates each analysis step but also it reduces the amount of low-quality sequence reads. Removal of such low-quality reads decrease time and cost of computation analysis and also reliable and high-quality results are obtained.

In any downstream analysis of NGS data, false-positive and false-negative results are produced due to:
experimental factors: like sample contamination or PCR errors,
sequencing factors: these include quality of sequencing and data contamination caused due to index hopping while splitting data, and/or
parameters of analysis software factors: this includes alignment software or precise type of parameter adjustment of downstream personalized analysis.
   The raw sequences generated after sequencing not only contain the sequences of interest, e.g. of target RNA molecules, but they also have sequence biases (for instance through systematic effects like Poisson sampling) and complex artefacts which are generated due to sequencing and experimental steps. These sequence biases and artefacts affect and interfere with precise read alignments which influence the genotyping and variant calling. Therefore, in order to increase the reliability and quality of downstream analysis and reduce the amount of required computational resources, the pre-processing of raw sequence reads is a necessity.

The present inventors found that 5′end and/or 3′end additions to target RNA molecules, e.g. produced or occurring during sequencing processes, e.g. next generation sequencing processes, are misleading and falsify the raw data results. Specifically, adaptor contaminated sequence reads need to be eliminated/excluded. The present inventors found that by elimination/exclusion of adaptor contaminated sequence reads the quality of raw data can be improved. Advantageously, the 5′end and/or 3′end additions to target RNA molecules can be detected using the at least 3 artificial small RNA molecules (spike-ins) comprised in/from the composition of the first aspect of the present invention. The presence of 5′end and/or 3′end additions identified with respect to the at least 3 artificial small RNA molecules (spike-ins) comprised in/from the composition of the first aspect of the present invention is indicative for the presence of 5′end and/or 3′end additions in the target RNA molecules. In this way, the (raw) data processing can be controlled, specifically improved by excluding RNAs that are not of biological origin and should not be used for biological interpretations of the data.

In a fourth aspect, the present invention relates to a method for examining a sample comprising the step of:
evaluating a sample with respect to the at least 3 RNA molecules (e.g. 3 or 4 RNA molecules) comprised in/from the composition of the first aspect.

In one embodiment, the sample is a mixture of a biological material with the composition of the first aspect.

Preferably, the biological material is tissue or a body fluid. More preferably, the body fluid is blood. Even more preferably, the blood is whole blood or a blood fraction. Particularly, the blood fraction is selected from the group consisting of a blood cell fraction and plasma or serum. The blood cell fraction represents the cellular portion of (whole) blood. Plasma and serum represent the acellular portion of (whole) blood. More particularly, the blood cell fraction comprises erythrocytes, leukocytes, or thrombocytes, the blood cell fraction is a fraction of erythrocytes, leukocytes, or thrombocytes, or the blood cell fraction is a mixture of erythrocytes, leukocytes, and thrombocytes.

In one alternative embodiment, the sample is based on/derived from a mixture of a biological material with the composition of the first aspect.

Specifically, the sample based on/derived from a mixture of a biological material with the composition of the first aspect is a processed sample. More specifically, the processed sample is a lysed sample, an extracted sample, an amplified sample, a sequenced sample, or a library prepped sample.
The processed sample may be obtained by adding the composition of the first aspect to the biological material, mixing the composition of the first aspect with the biological material, and further processing the sample.
Preferably, the processed sample is obtained by mixing a biological material with the composition of the first aspect and further processing the mixture obtained thereby.

In order to evaluate the sample with respect to the at least 3 RNA molecules (e.g. 3 or 4 RNA molecules) comprised in/from the composition of the first aspect, the level of said at least 3 RNA molecules (e.g. 3 or 4 RNA molecules) in the sample is preferably determined.

In one preferred embodiment, the evaluation comprises determining whether the at least 3 RNA molecules (e.g. 3 or 4 RNA molecules) show a characteristic distribution, or determining whether the characteristic distribution of the RNA molecules in the sample matches the expected characteristic distribution.

As mentioned above, the at least 3 RNA molecules (e.g. 3 or 4 RNA molecules) comprised in the composition of the first aspect have a characteristic distribution, specifically with respect to their amounts. Particularly, the at least 3 RNA (e.g. 3 or 4 RNA molecules) molecules are comprised in the composition of the first aspect in different amounts. More particularly, the at least 3 RNA molecules (e.g. 3 or 4 RNA molecules) are comprised in the composition of the first aspect in a gradient of defined amounts.

In order to determine, whether the at least 3 RNA molecules (e.g. 3 or 4 RNA molecules) show a characteristic distribution, the level of said at least 3 RNA molecules (e.g. 3 or 4 RNA molecules) in the sample is preferably determined.

If the characteristic distribution is given, the sample is further processed and/or analysed. Particularly, the characteristic distribution is given if
the at least 3 RNA molecules are present in their expected level,
the at least 3 RNA molecules are present at their expected order/rank (defined by the relation of
the levels, in particular amounts, of the at least 3 RNA molecules), and/or
the at least 3 RNA molecules are present in their expected linearity.

If the characteristic distribution is not given, the sample is not further processed and/or analysed. It is discarded.

Particularly, the characteristic distribution is not given if
the at least 3 RNA molecules are not present in their expected level,
the at least 3 RNA molecules are not present at their expected order/rank (defined by the relation of the levels, in particular amounts, of the at least 3 RNA molecules), and/or
the at least 3 RNA molecules are not present in their expected linearity.

With expected level, the level which is expected for the specific RNA molecule amount added to the sample in subsequent sample processing and/or analysis is meant. As mentioned above, the RNA molecule is added to the sample or comprised in the sample in a specific amount. The amount of the RNA molecule may correspond to/correlate with any expected level of the RNA molecule which can be measured during subsequent sample processing and/or analysis. Specifically, the amount of the RNA molecule may correspond to/correlate with specific read counts (reads per million (RPM)) in a next generation sequencing assay and/or the amount of the RNA molecule may correspond to/correlate with a specific cycle threshold (Ct) in a real-time PCR experiment. In particular, the cycle threshold (Ct) is the number of cycles in a real-time PCR that are required to exceed a previously defined threshold in the measurement signal (e.g. a fluorescence signal) of the amplified DNA. The more DNA (RNA) was already present in a sample solution before PCR, the fewer amplification cycles are required to reach the corresponding threshold value.

For example, the RNA molecule having a nucleotide sequence according to SEQ ID NO: 1 added to the sample or comprised in the composition of the first aspect in an amount of about 3400 amol will result in a predicted Ct value of about 26 (Ct mean of about 24.3), the RNA molecule having a nucleotide sequence according to SEQ ID NO: 2 added to the sample or comprised in the composition of the first aspect in an amount of about 725 amol will result in a predicted Ct value of about 29.3 (Ct mean of about 27.1), the RNA molecule having a nucleotide sequence according to SEQ ID NO: 3 added to the sample or comprised in the composition of the first aspect in an amount of about 20 amol will result in a predicted Ct value of about 35.9 (Ct mean of about 32), and/or the RNA molecule having a nucleotide sequence according to SEQ ID NO: 4 added to the sample or comprised in the composition of the first aspect in an amount of about 7 amol will result in a predicted Ct value of about 39.2 (Ct mean of about 33.6).
For example, the RNA molecule having a nucleotide sequence according to SEQ ID NO: 1 added to the sample or comprised in the composition of the present invention in an amount of about 3400 amol will result in a log2 RPM of about 11.972 or in read counts of about 30628, the RNA molecule having a nucleotide sequence according to SEQ ID NO: 2 added to the sample or comprised in the composition of the first aspect in an amount of about 725 amol will result in a log2 RPM of about 10.926 or in read counts of about 5554, the RNA molecule having a nucleotide sequence according to SEQ ID NO: 3 added to the sample or comprised in the composition of the first aspect in an amount of about 20 amol will result in a log2 PRM of about 6.559 or in read counts of about 491, and/or the RNA molecule having a nucleotide sequence according to SEQ ID NO: 4 added to the sample or comprised in the composition of the first aspect in an amount of about 7 amol will result in a log2 RPM of about 4.061 or in read counts of about 94.

With expected order/rank, the order/rank which is expected for the specific RNA molecules added to the sample in subsequent sample processing and/or analysis is meant. As mentioned above, the RNA molecules added to the sample differ in their respective amounts. Thus, in case 3 RNA molecules are added to a sample or comprised in the composition of the first aspect, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules. In case 4 RNA molecules are added to a sample or comprised in the composition of the first aspect, the first RNA molecule (e.g. the RNA molecule having a nucleotide sequence according to SEQ ID NO: 1) is present in the largest amount, the second RNA molecule (e.g. the RNA molecule having a nucleotide sequence according to SEQ ID NO: 2) is present in a smaller amount than the first RNA molecule, and the third RNA molecule (e.g. the RNA molecule having a nucleotide sequence according to SEQ ID NO: 3) is present in a smaller amount than the first and second RNA molecules, and the fourth RNA molecule (e.g. the RNA molecule having a nucleotide sequence according to SEQ ID NO: 4) is present in a smaller amount than the first, second, and third RNA molecules.

This expected order/rank, representing the initial state/condition, must be recovered during sample processing and/or analysis. In this respect, it should be noted that the amount of the RNA molecule comprised in a specific order/rank in the sample or composition may correspond to/correlate with any expected level of the RNA molecule which can be measured during subsequent sample processing and/or analysis. For example, the amount of the RNA molecule may correspond to/correlate with specific read counts in a next generation sequencing assay and/or the amount of the RNA molecule may correspond to/correlate with a specific cycle threshold (Ct) in a real-time PCR experiment.

With expected linearity, the linearity which is expected for the specific RNA molecule added to the sample in subsequent sample processing and/or analysis is meant.

This expected linearity, representing the initial state/condition, must be recovered during sample processing and/or analysis.

Thus, only those samples of which the analysis resulted in the recovery of the RNA molecules or surrogates thereof in their expected level, order, and/or linearity are used for downstream analysis and/or further processing.

To determine, whether the RNA molecules are present in their expected order and/or linearity, statistical analysis are performed with data (e.g. Ct values or RPM) relating to said RNA molecules. Said statistical analysis include, but are not limited to, Spearman (rank) correlation analysis and/or Pearson correlation analysis.

For example, in order to determine, whether the RNA molecules are present in their expected order, the Spearman's rank correlation coefficient (Spearman's ρ) is preferably determined. In addition, in order to determine, whether the RNA molecules are present in their expected linearity, the Pearson's correlation coefficient (Pearson's r) is preferably determined.

Preferably, the RNA molecules are present in their expected order when the Spearman's rank correlation coefficient (Spearman's ρ) is ≥0.95. In this case, the sample is further processed/analysed. Thus, a Spearman's rank correlation coefficient (Spearman's ρ) of <0.95 leads to the discard of the sample. In other words, such a sample is not further processed/analyzed.

Preferably, the RNA molecules are present in their expected linearity when the Pearson's correlation coefficient (Pearson's r) of ≥0.66. In this case, the sample is further processed/analysed. Thus, a Pearson's correlation coefficient (Pearson's r) of <0.66 leads to the discard of the sample. In other words, such a sample is not further processed/analyzed.

The further processing may encompass lysing the sample, extracting the sample, amplifying the sample, sequencing the sample, and/or preparing a library from the sample. Specifically, the further processing encompasses lysing the cells to release the nucleotide sequences comprised in the sample, extracting the nucleotide sequences comprised in the sample, amplifying the nucleotide sequences comprised in the sample, sequencing the nucleotide sequences comprised in the sample, and/or preparing a library from the nucleotide sequences comprised in the sample. Especially, the nucleotide sequences are ribonucleotide sequences. Preferably, the ribonucleotide sequences belong to target RNA molecules. More preferably, the target RNA molecules are small RNA molecules. Even more preferably, the small RNA molecules are non-coding small RNA molecules. Most preferably, the non-coding small RNA molecules are miRNA and/or isomiR molecules.

In this respect, it should be noted that the artificial, exogenous RNA molecules added to the biological material are processed like the endogenous target RNA molecules comprised in the biological material.

RNAs such as small RNAs in biological samples like biofluids play an important role as prognostic and diagnostic biomarkers for many human disease states. However, accurate analysis of these RNAs in biological samples such as biofluids is of great importance with regard to the meaningfulness of these data in the biomarker field.

In the method of the present invention relating to the examination of a sample, a sample is evaluated with respect to the at least 3 RNA molecules comprised in/from the composition described herein, wherein the at least 3 RNA molecules are selected from a group consisting of RNA molecules having a nucleotide sequence according to SEQ ID NO: 1 to SEQ ID NO: 4. The at least 3 RNA molecules are part of the sample. They have been added to the sample (as spike-ins).

In this method, it is alternatively or additionally verified/analysed whether the at least 3 RNA molecules having a nucleotide sequence according to SEQ ID NO: 1 to SEQ ID NO: 4 comprise 5′end and/or 3′end additions.

In this respect, it will be appreciated that the term “RNA molecule” may refer to the RNA molecule itself as well as to surrogates thereof, for example, amplification products (e.g. cDNA derived therefrom).

Thus, in one preferred embodiment, the evaluation comprises identifying 5′end and/or 3′end additions of the at least 3 RNA molecules (e.g. 3 or 4 RNA molecules) comprised in/from the composition of the first aspect.

Preferably, the 5′end and/or 3′end additions have a length of at least 5 nucleotides. More preferably, the 5′end and/or 3′end additions have a length of between 5 and 30 nucleotides, even more preferably between 5 and 20 nucleotides, and still even more preferably between 7 and 15 nucleotides, e.g. 5, 6, 7, 8, 9, 10, 11, 12, 13, 14, 15, 16, 17, 18, 19, 20, 21, 22, 23, 24, 25, 26, 27, 28, 29, or 30 nucleotides. The 5′end additions can also be designated as prefixes, the 3′end additions can also be designated as suffixes, and the additions at the 5′end as well as 3′end can also be designated as affixes.

Specifically, the at least 5 nucleotides extend beyond the original length of the at least 3 RNA molecules.

Prefixes, suffixes, as well as affixes can also extend the original length of the RNA molecules by 1, 2, 3, or 4 nucleotides. However, to reduce the risk of falsely identifying RNA molecules as having 5′end and/or 3′end additions relevant in this context, a minimum length of 5 nucleotides is required/recommended herein.

The 5′end and/or 3′end additions to the at least 3 RNA molecules comprised in/from the composition of the first aspect can easily be determined using sequencing and/or sequence alignment methods. As the sequence of the artificial small RNA molecules (spike-ins) is known, the identification/analysis of the added nucleotides is no practical problem and a standard procedure for the skilled person. As mentioned above, to reduce the risk of falsely identified RNA molecules as having 5′end and/or 3′end additions relevant in this context, a minimum length of 5 nucleotides is required to be designated herein as 5′end addition and/or 3′end addition.

The 5′end and/or 3′ end additions may be the result of RNA molecule/adapter fusion, RNA molecule/RNA molecule fusion, or adapter/adapter fusion. The adapter may be any non-biological RNA/DNA sequence that is intentionally added to the 5′end or 3′end of an RNA/a cDNA molecule, e.g. as part of a sequencing such as NGS method. This includes any free-floating adapter molecules that may fuse with RNA in an unintended manner. An adapter can also be a combination/fusion of multiple individual adapters and indexing RNAs. Indexing RNAs are used as UMI (unique molecular identifier) for assigning RNA molecules to their sample of origin during multiplexed sequencing, i.e. sequencing multiple samples at the same time. UMI sequences are short unspecific (random) sequences of a predefined length, e.g. 12 nucleotides, but can also vary in length.

The sample evaluated in the above embodiment preferably comprises target RNA molecules, e.g. comprised in a biological material such as whole blood or derived from a biological material such as whole blood.

Thus, in a next step, it is preferably determined whether the 5′end and/or 3′end additions identified with respect to the at least 3 RNA molecules comprised in/from the composition of the first aspect (artificial small RNA molecules/spike-ins) are also found in the target RNA molecules or surrogate thereof (e.g. cDNA molecules) which are part of/comprised in the sample. In other words, the presence of 5′end and/or 3′end additions identified with respect to the at least 3 RNA molecules comprised in/from the composition of the first aspect (artificial small RNA molecules/spike-ins) is preferably indicative for the presence of 5′end and/or 3′end additions in the target RNA molecules or surrogate thereof (e.g. cDNA molecules).

Preferably, the 5′end and/or 3′ end additions take place/occur during:
ligation of 5′adapters and/or 3′adapters to (denatured) (target) RNA molecules, e.g. using/with a double stranded RNA ligase such as T4 RNA ligase 2 (Rnl2) or a Kod1 ligase,
reverse transcription of (target) RNA molecules (having 5′adapters and/or 3′adapters ligated thereon, also designated as ligation products) into cDNA molecules, e.g. using/with a reverse transcriptase (RT) such as Maxima H-RT or Tth polymerase,
amplification of (said) cDNA molecules, e.g. via polymerase chain reaction (PCR), and/or
sequencing such as next generation sequencing of (said) cDNA molecules.

More preferably, the 5′end and/or 3′ end additions take place/occur during library preparation process for next generation sequencing or during the next generation sequencing process.

Even more preferably, the next generation sequencing process preferably encompasses:
ligation of 5′adapters and/or 3′adapters to (denatured) (target) RNA molecules, e.g. using/with a double stranded RNA ligase such as T4 RNA ligase 2 (Rnl2) or a Kod1 ligase,
reverse transcription of (target) RNA molecules (having 5′adapters and/or 3′adapters ligated thereon, also designated as ligation products) into cDNA molecules, e.g. using/with a reverse transcriptase (RT) such as Maxima H-RT or Tth polymerase,
amplification of (said) cDNA molecules, e.g. via polymerase chain reaction (PCR), and/or
sequencing of (said) cDNA molecules.

Still even more preferably, the 5′end and/or 3′end additions are selected from the group consisting of additions having a nucleotide sequence according to CGATC (SEQ ID NO: 10), GGGGC (SEQ ID NO: 11), ACGATC (SEQ ID NO: 12), GGGCGT (SEQ ID NO: 13), CGGCGG (SEQ ID NO: 14), GGGGCG (SEQ ID NO: 15), GACGATC (SEQ ID NO: 16), GGGGCGT (SEQ ID NO: 17), GGGCGTG (SEQ ID NO: 18), GGGGGCG (SEQ ID NO: 19), GGGGGTG (SEQ ID NO: 20), GGGGCGTG (SEQ ID NO: 21), CGGGGCGG (SEQ ID NO: 22), GGGAGGCC (SEQ ID NO: 23), GGAGGCGT (SEQ ID NO: 24), GGGCGTGG (SEQ ID NO: 25), TGGAGGCG (SEQ ID NO: 26), CGACGATC (SEQ ID NO: 27), GGGGCGTT (SEQ ID NO: 28), GGGCGTGT (SEQ ID NO: 29), GGGGGCGT (SEQ ID NO: 30), GGGAGCCA (SEQ ID NO: 31), GGGGGTGT (SEQ ID NO: 32), GGAGGCCC (SEQ ID NO: 33), CCGACGATC (SEQ ID NO: 34), GGGGGCGTG (SEQ ID NO: 35), TACCTGGTT (SEQ ID NO: 36), TGGAGGCGT (SEQ ID NO: 37), GGGCGTGGG (SEQ ID NO: 38), CGGCGGCGG (SEQ ID NO: 39), GGGGGTGTA (SEQ ID NO: 40), GGGGGCGTT (SEQ ID NO: 41), GGCTGGGCG (SEQ ID NO: 42), TCGGGGCGG (SEQ ID NO: 43), GGGGCGTGG (SEQ ID NO: 44), GGGGAGCCA (SEQ ID NO: 45), GGGAGGCCC (SEQ ID NO: 46), CGGAGGGCGG (SEQ ID NO: 47), GTCCGCGATC (SEQ ID NO: 48), GTCGACGATC (SEQ ID NO: 49), CGGGCGGATC (SEQ ID NO: 50), TGGAGGCGTG (SEQ ID NO: 51), TCCGACGATC (SEQ ID NO: 52), GGGGCGTGGG (SEQ ID NO: 53), AAGCGGGGCT (SEQ ID NO: 54), CGGGGAGCCA (SEQ ID NO: 55), GTCCGACGATC (SEQ ID NO: 56), TCGGAGGGCGG (SEQ ID NO: 57), AGTCCGACGATC (SEQ ID NO: 58), AAGCGGGGCTGG (SEQ ID NO: 59), GTCCGACGGATC (SEQ ID NO: 60), TCGGGCTGGGGC (SEQ ID NO: 61), TACCTGGTTGAT (SEQ ID NO: 62), TCGGGGCGGCGG (SEQ ID NO: 63), CAGTCCGACGATC (SEQ ID NO: 64), TACCTGGTTGATC (SEQ ID NO: 65), TCGGGCTGGGGCG (SEQ ID NO: 66), TGGAGGCGTGGGT (SEQ ID NO: 67), ACAGTCCGACGATC (SEQ ID NO: 68), GGTCGGGCTGGGGC (SEQ ID NO: 69), CGGAAGCGTGCTGGG (SEQ ID NO: 70), GGTCGGGCTGGGGCG (SEQ ID NO: 71), TACAGTCCGACGATC (SEQ ID NO: 72), CGGAAGCGTGCTGGGC (SEQ ID NO: 73), CTACAGTCCGACGATC (SEQ ID NO: 74), TCTACAGTCCGACGATC (SEQ ID NO: 75), CGGAAGCGTGCTGGGCCC (SEQ ID NO: 76), TCGGGGCGGCGGCGGCGG (SEQ ID NO: 77), TTCTACAGTCCGACGATC (SEQ ID NO: 78), TAGCAGCACATCATGGTT (SEQ ID NO: 79), GGATCATTA (SEQ ID NO: 80), GGGGCGTGGG (SEQ ID NO: 81), and TGGAGGCGTGGGT (SEQ ID NO: 82).

Specifically, the determination of the 5′end and/or 3′end additions to the (target) RNA molecules encompasses the analysis whether the (target) RNA molecules are at least in part sequence identical with adapter sequences used in the sequencing, preferably next generation sequencing, process.

More specifically, the adapter sequences used in the process of next generation sequencing are selected from the group consisting of TGGAATTCTCGGGTGCCAAGG (SEQ ID NO: 83), GTTCAGAGTTCTACAGTCCGACGATC (SEQ ID NO: 84), TGGAATTCTCGGGTGCCAAGG (SEQ ID NO: 85), GAATTCCACCACGTTCCCGTGG (SEQ ID NO: 86), AATGATACGGCGACCACCGAGATCTACACGTTCAGAGTTCTACAGTCCGA (SEQ ID NO: 87), CAAGCAGAAGACGGCATACGAGAT (SEQ ID NO: 88), GTGACTGGAGTTCCTTGGCACCCGAGAATTCCA (SEQ ID NO: 89), CAAGCAGAAGACGGCATACGA (SEQ ID NO: 90), GTTCAGAGTTCTACAGTCCGACGATC (SEQ ID NO: 91), TCGTATGCCGTCTTCTGCTTGT (SEQ ID NO: 92), ATCTCGTATGCCGTCTTCTGCTTG (SEQ ID NO: 93), CAAGCAGAAGACGGCATACGA (SEQ ID NO: 94), AATGATACGGCGACCACCGACAGGTTCAGAGTTCTACAGTCCGA (SEQ ID NO: 95), CGACAGGTTCAGAGTTCTACAGTCCGACGATC (SEQ ID NO: 96), AAAAAAAAAAAAAAAAAAAAAAAAAAAAAAAAAAAAAAAAAAAAAA (SEQ ID NO: 97), AGATCGGAAGAGCACACGTCTGAACTCCAGTCA (SEQ ID NO: 98), AGATCGGAAGAGCGTCGTGTAGGGAAAGAGTGT (SEQ ID NO: 99), GATCGGAAGAGCACACGTCTGAACTCCAGTCAC (SEQ ID NO: 100), ATCTCGTATGCCGTCTTCTGCTTG (SEQ ID NO: 101), AATGATACGGCGACCACCGAGATCTACAC (SEQ ID NO: 102), and ACACTCTTTCCCTACACGACGCTCTTCCGATCT (SEQ ID NO: 103).

Process control, quality control and pre-processing of data are important for data analysis because raw data produced after sequencing must be processed so that the results should not have false positive and false negative results. Pre-processing of data not only evaluates each analysis step but also it reduces the amount of low-quality sequence reads. Removal of such low-quality reads decrease time and cost of computation analysis and also reliable and high-quality results are obtained.

In any downstream analysis of NGS data, false-positive and false-negative results are produced due to:
experimental factors: like sample contamination or PCR errors,
sequencing factors: these include quality of sequencing and data contamination caused due to index hopping while splitting data, and/or
parameters of analysis software factors: this includes alignment software or precise type of parameter adjustment of downstream personalized analysis.
   The raw sequences generated after sequencing not only contain the sequences of interest, e.g. of target RNA molecules, but they also have sequence biases (for instance through systematic effects like Poisson sampling) and complex artefacts which are generated due to sequencing and experimental steps. These sequence biases and artefacts affect and interfere with precise read alignments which influence the genotyping and variant calling. Therefore, in order to increase the reliability and quality of downstream analysis and reduce the amount of required computational resources, the pre-processing of raw sequence reads is a necessity.

The present inventors found that 5′end and/or 3′end additions to (target) RNA molecule, e.g. produced or occurring during sequencing, e.g. next generation sequencing, processes are misleading and falsify the raw data results. Specifically, adaptor contaminated sequence reads need to be eliminated/excluded. The present inventors found that by elimination/exclusion of adaptor contaminated sequence reads the quality of raw data can be improved.

Accordingly, target RNA molecules with 5′end and/or 3′end additions comprised in the sample are excluded from further analyses/are not further used. Alternatively or additionally, data from target RNA molecules with 5′end and/or 3′end additions comprised in the sample are excluded from further analyses/are not further used or do not form part of a data set, preferably raw data set. For example, the data concerning these target RNA molecules are simply excluded from the (raw) data set.

Preferably, the target RNA molecules are small RNA molecules. More preferably, the small RNA molecules are non-coding small RNA molecules. Even more preferably, the non-coding small RNA molecules are miRNA and/or isomiR molecules.

The sample used in the above embodiment is preferably a lysed sample, an extracted sample, an amplified sample, and/or a sequenced sample. The sample lysis encompasses the lysis of cells comprised in the sample and is required to release the (target) RNA molecules comprised therein. The extraction of a sample is required to extract the (target) RNA molecules comprised therein. The amplification of a sample is required to amplify the (target) RNA molecules comprised therein. The sequencing of a sample is required to sequence the (target) RNA molecules comprised therein.

For example, as sample a biological sample such as whole blood sample is provided. In a first step, the sample, specifically the cells comprised in the sample, are lysed to release the (target) RNA molecules comprised therein. Afterwards, cDNA is produced from the (target) RNA molecules using adapters ligated to the denatured (target) RNA molecules via reverse transcription. The achieved cDNA molecules are subsequently amplified to finally allow sequencing such as next generation sequencing of said cDNA molecules.
In any of the above processes, 5′end and/or 3′end additions may be added to the (target) RNA molecules which need to be identified and (target) RNA molecules having these 5′end and/or 3′end additions need to be excluded from further analysis or raw data assessment. This improves data quality.

The analysis of RNA expression profiles involves several intricate steps. (1) RNA, including small RNA, must be extracted from a biological source, e.g. blood or saliva, without distorting the original relative abundances of the RNA (linearity must be preserved). (2) The small RNA abundances must be reliably quantified. Since both steps can inconsistently introduce bias (e.g. through the presence of inhibitors of cDNA synthesis) it is crucial to standardize but also monitor both the efficiency of RNA extraction and detection. Addressing these challenges can be achieved in several ways.

The present inventors have designed and optimized a universal spike-in system of artificial small RNA molecules that broadly reflects endogenous miRNA behaviour during RNA extraction and detection. This concoction of artificial small RNA molecules is then added to biological samples such as clinical samples at the processing start and serves as an end-to-end quality control measure: only those samples of which the analysis resulted in the recovery of the artificial small RNA molecules in their expected level, order, and linearity qualify are used for downstream analysis.

Thus, in a fifth aspect, the present invention relates to a method for optimized processing of biological samples comprising the step of:
carrying out the method of the fourth aspect.

In a sixth aspect, the present invention relates to a method for optimized RNA preparation from or RNA analysis of biological samples comprising the step of:
carrying out the method of the fourth aspect.

In a seventh aspect, the present invention relates to a method for improving (RNA) data set quality comprising the step of:
carrying out the method of the fourth aspect.

Process control, quality control and pre-processing of data are important for data analysis because data such as raw data produced after sequencing must be processed so that the results should not have false positive and false negative results. Pre-processing of data not only evaluates each analysis step but also it reduces the amount of low-quality sequence reads. Removal of such low-quality reads decrease time and cost of computation analysis and also reliable and high-quality results are obtained.

In any downstream analysis of NGS data, false-positive and false-negative results are produced due to:
experimental Factors: like sample contamination or PCR errors,
sequencing Factors: these include quality of sequencing and data contamination caused due to index hopping while splitting data, and/or
parameters of analysis software factors: this includes alignment software or precise type of parameter adjustment of downstream personalized analysis.
   The raw sequences generated after sequencing not only contain the sequences of interest, e.g. of target RNA molecules, but they also have sequence biases (for instance through systematic effects like Poisson sampling) and complex artefacts which are generated due to sequencing and experimental steps. These sequence biases and artefacts affect and interfere with precise read alignments which influence the genotyping and variant calling. Therefore, in order to increase the reliability and quality of downstream analysis and reduce the amount of required computational resources, the pre-processing of raw sequence reads is a necessity.

The present inventors found that 5′end and/or 3′end additions to (target) RNA molecule, e.g. produced or occurring during sequencing processes, e.g. next generation sequencing processes, are misleading and falsify the raw data results. Specifically, adaptor contaminated sequence reads need to be eliminated/excluded. The present inventors found that by elimination/exclusion of adaptor contaminated sequence reads the quality of data such s raw data can be improved.

In an eight aspect, the present invention relates to a method for improving (RNA) data set quality comprising the steps of:
(i) determining the sequence of (target) RNA molecules (specifically of surrogates such as cDNA molecules derived therefrom) in a sample,
(ii) determining 5′end and/or 3′end additions to the (target) RNA molecules, which are not part of the (target) RNA molecules in naturally occurring form, and
(iii) excluding (target) RNA molecules having 5′end and/or 3′end additions from (RNA) data set analysis/removing (target) RNA molecules having 5′end and/or 3′end additions from the (RNA) data set.

In step (i) of the above method, the sequence of the RNA molecules (specifically of surrogates such as cDNA molecules derived therefrom) in the sample is determined. The sequence of the RNA molecules (specifically of surrogates such as cDNA molecules derived therefrom) in a sample may be determined by any method known to the skilled person. Known sequencing methods include, but are not limited to, sanger sequencing, capillary electrophoreses and fragment analysis, or next-generation sequencing (NGS). Preferably, the sequence of the RNA molecules (specifically of surrogates such as cDNA molecules derived therefrom) in a sample is determined via NGS.

Specifically, the determination of the sequence of RNA molecules, particularly via next generation sequencing, in the sample encompasses:
denaturation of (target) RNA molecules and ligation of 5′adapters and/or 3′adapters to the denatured (target) RNA molecules, e.g. using/with a double stranded RNA ligase such as T4 RNA ligase 2 (Rnl2) or a Kodl ligase,
reverse transcription of RNA molecules (having 5′adapters and/or 3′adapters ligated thereon, also designated as ligation products) into cDNA molecules, e.g. using/with a reverse transcriptase (RT) such as Maxima H-RT or Tth polymerase,
amplification of (said) cDNA molecules, e.g. via polymerase chain reaction (PCR), and/or
sequencing, particularly next generation sequencing, of (said) cDNA molecules.
   The PCR is preferably selected from the group consisting of real-time PCR (quantitative PCR or qPCR), preferably TaqMan qPCR, multiplex PCR, nested PCR, high fidelity PR, fast PCR, hot start PCR, and GC-rich PCR.

In step (ii) of the above method, 5′end and/or 3′end additions to the (target) RNA molecules, which are not part of the (target) RNA molecules in naturally occurring form, are determined.

Naturally occurring RNA molecules are in the form in which they occur in nature or in a natural environment, e.g. bodily fluid such as whole blood, or tissue. These RNA molecules may, however, be further processed, e.g. into surrogates thereof such as cDNA molecules. In this case, they have a natural origin. Naturally occurring RNA molecules have an endogenous origin in a biological material/sample. Said RNA molecules are originally part of said biological material/sample.

The 5′end and/or 3′end additions have preferably a length of at least 5 nucleotides. More preferably, the 5′end and/or 3′end additions have a length of between 5 and 30 nucleotides, even more preferably between 5 and 20 nucleotides, and still even more preferably between 7 and 15 nucleotides, e.g. 5, 6, 7, 8, 9, 10, 11, 12, 13, 14, 15, 16, 17, 18, 19, 20, 21, 22, 23, 24, 25, 26, 27, 28, 29, or 30 nucleotides. The 5′end additions can also be designated as prefixes, the 3′end additions can also be designated as suffixes, and the additions at the 5′end as well as 3′end can also be designated as affixes. Specifically, the at least 5 nucleotides extend beyond the length of the RNA molecules in naturally occurring form.

Prefixes, suffixes, as well as affixes can also be 1, 2, 3, or 4 nucleotides long. However, to reduce the risk of falsely identifying (target) RNA molecules as having 5′end and/or 3′end additions, a minimum length of 5 nucleotides is required/recommended herein.

The 5′end and/or 3′end additions to the (target) RNA molecules comprised in the sample can be determined using sequencing and and comparing the 5′/3′ ends of the sequence with the sequences of the additions.

The 5′end and/or 3′end additions are the result of RNA molecule/adapter fusion, RNA molecule/RNA molecule fusion, or incomplete adapter processing/removal.

The adapter may be any non-biological RNA/DNA sequence that is intentionally added to the 5′end or 3′end of a biological (target) RNA/cDNA molecule (originating from the sample to be sequenced) as part of the design of a sequencing such as NGS method. This includes any free-floating adapter molecules that may fuse with biological RNA in an unintended manner. An adapter can also be a combination/fusion of multiple individual adapters and indexing RNAs. Indexing RNAs are used as UMI (unique molecular identifier) for assigning RNA molecules to their sample of origin during multiplexed sequencing, i.e. sequencing multiple samples at the same time. UMI sequences are short unspecific (random) sequences of a predefined length, e.g. 12 nucleotides, but can also vary in length.

The 5′end and/or 3′ end additions are preferably selected from the group consisting of additions having a nucleotide sequence according to CGATC (SEQ ID NO: 10), GGGGC (SEQ ID NO: 11), ACGATC (SEQ ID NO: 12), GGGCGT (SEQ ID NO: 13), CGGCGG (SEQ ID NO: 14), GGGGCG (SEQ ID NO: 15), GACGATC (SEQ ID NO: 16), GGGGCGT (SEQ ID NO: 17), GGGCGTG (SEQ ID NO: 18), GGGGGCG (SEQ ID NO: 19), GGGGGTG (SEQ ID NO: 20), GGGGCGTG (SEQ ID NO: 21), CGGGGCGG (SEQ ID NO: 22), GGGAGGCC (SEQ ID NO: 23), GGAGGCGT (SEQ ID NO: 24), GGGCGTGG (SEQ ID NO: 25), TGGAGGCG (SEQ ID NO: 26), CGACGATC (SEQ ID NO: 27), GGGGCGTT (SEQ ID NO: 28), GGGCGTGT (SEQ ID NO: 29), GGGGGCGT (SEQ ID NO: 30), GGGAGCCA (SEQ ID NO: 31), GGGGGTGT (SEQ ID NO: 32), GGAGGCCC (SEQ ID NO: 33), CCGACGATC (SEQ ID NO: 34), GGGGGCGTG (SEQ ID NO: 35), TACCTGGTT (SEQ ID NO: 36), TGGAGGCGT (SEQ ID NO: 37), GGGCGTGGG (SEQ ID NO: 38), CGGCGGCGG (SEQ ID NO: 39), GGGGGTGTA (SEQ ID NO: 40), GGGGGCGTT (SEQ ID NO: 41), GGCTGGGCG (SEQ ID NO: 42), TCGGGGCGG (SEQ ID NO: 43), GGGGCGTGG (SEQ ID NO: 44), GGGGAGCCA (SEQ ID NO: 45), GGGAGGCCC (SEQ ID NO: 46), CGGAGGGCGG (SEQ ID NO: 47), GTCCGCGATC (SEQ ID NO: 48), GTCGACGATC (SEQ ID NO: 49), CGGGCGGATC (SEQ ID NO: 50), TGGAGGCGTG (SEQ ID NO: 51), TCCGACGATC (SEQ ID NO: 52), GGGGCGTGGG (SEQ ID NO: 53), AAGCGGGGCT (SEQ ID NO: 54), CGGGGAGCCA (SEQ ID NO: 55), GTCCGACGATC (SEQ ID NO: 56), TCGGAGGGCGG (SEQ ID NO: 57), AGTCCGACGATC (SEQ ID NO: 58), AAGCGGGGCTGG (SEQ ID NO: 59), GTCCGACGGATC (SEQ ID NO: 60), TCGGGCTGGGGC (SEQ ID NO: 61), TACCTGGTTGAT (SEQ ID NO: 62), TCGGGGCGGCGG (SEQ ID NO: 63), CAGTCCGACGATC (SEQ ID NO: 64), TACCTGGTTGATC (SEQ ID NO: 65), TCGGGCTGGGGCG (SEQ ID NO: 66), TGGAGGCGTGGGT (SEQ ID NO: 67), ACAGTCCGACGATC (SEQ ID NO: 68), GGTCGGGCTGGGGC (SEQ ID NO: 69), CGGAAGCGTGCTGGG (SEQ ID NO: 70), GGTCGGGCTGGGGCG (SEQ ID NO: 71), TACAGTCCGACGATC (SEQ ID NO: 72), CGGAAGCGTGCTGGGC (SEQ ID NO: 73), CTACAGTCCGACGATC (SEQ ID NO: 74), TCTACAGTCCGACGATC (SEQ ID NO: 75), CGGAAGCGTGCTGGGCCC (SEQ ID NO: 76), TCGGGGCGGCGGCGGCGG (SEQ ID NO: 77), TTCTACAGTCCGACGATC (SEQ ID NO: 78), TAGCAGCACATCATGGTT (SEQ ID NO: 79), GGATCATTA (SEQ ID NO: 80), GGGGCGTGGG (SEQ ID NO: 81), and TGGAGGCGTGGGT (SEQ ID NO: 82).

In case the above 5′end and/or 3′ end additions are detected or identified in (target) RNA molecules or surrogates thereof, said (target) RNA molecules or surrogates thereof are excluded in step (iii) of the above method from (RNA) data set analysis or said (target) RNA molecules or surrogates thereof are removed in step (iii) of the above method from the (RNA) data set.

Specifically, the determination of 5′end and/or 3′end additions to the (target) RNA molecules encompasses the analysis whether the (target) RNA molecules are at least in part sequence identical with adapter sequences used in sequencing, preferably next generation sequencing.

More specifically, the adapter sequences used in the process of next generation sequencing are selected from the group consisting of TGGAATTCTCGGGTGCCAAGG (SEQ ID NO: 83), GTTCAGAGTTCTACAGTCCGACGATC (SEQ ID NO: 84), TGGAATTCTCGGGTGCCAAGG (SEQ ID NO: 85), GAATTCCACCACGTTCCCGTGG (SEQ ID NO: 86), AATGATACGGCGACCACCGAGATCTACACGTTCAGAGTTCTACAGTCCGA (SEQ ID NO: 87), CAAGCAGAAGACGGCATACGAGAT (SEQ ID NO: 88), GTGACTGGAGTTCCTTGGCACCCGAGAATTCCA (SEQ ID NO: 89), CAAGCAGAAGACGGCATACGA (SEQ ID NO: 90), GTTCAGAGTTCTACAGTCCGACGATC (SEQ ID NO: 91), TCGTATGCCGTCTTCTGCTTGT (SEQ ID NO: 92), ATCTCGTATGCCGTCTTCTGCTTG (SEQ ID NO: 93), CAAGCAGAAGACGGCATACGA (SEQ ID NO: 94), AATGATACGGCGACCACCGACAGGTTCAGAGTTCTACAGTCCGA (SEQ ID NO: 95), CGACAGGTTCAGAGTTCTACAGTCCGACGATC (SEQ ID NO: 96), AAAAAAAAAAAAAAAAAAAAAAAAAAAAAAAAAAAAAAAAAAAAAA (SEQ ID NO: 97), AGATCGGAAGAGCACACGTCTGAACTCCAGTCA (SEQ ID NO: 98), AGATCGGAAGAGCGTCGTGTAGGGAAAGAGTGT (SEQ ID NO: 99), GATCGGAAGAGCACACGTCTGAACTCCAGTCAC (SEQ ID NO: 100), ATCTCGTATGCCGTCTTCTGCTTG (SEQ ID NO: 101), AATGATACGGCGACCACCGAGATCTACAC (SEQ ID NO: 102), and ACACTCTTTCCCTACACGACGCTCTTCCGATCT (SEQ ID NO: 103).

In case the above adapter sequences or parts thereof are detected or identified in (target) RNA molecules or surrogates thereof, said (target) RNA molecules or surrogates thereof are excluded in step (iii) of the above method from (RNA) data set analysis or said (target) RNA molecules or surrogates thereof are removed in step (iii) of the above method from the (RNA) data set.

Particularly, the RNA data set analysis is RNA raw data set analysis/the RNA data set is a raw RNA data set.

Preferably, the (target) RNA molecules are small RNA molecules. More preferably, the small RNA molecules are non-coding small RNA molecules. Even more preferably, the non-coding small RNA molecules are miRNA and/or isomiR molecules.

The sample used in the above method may be a processed sample. Preferably, the sample used in the above method is a lysed sample, an extracted sample, an amplified sample, and/or a sequenced sample. The sample lysis encompasses the lysis of cells comprised in the sample and is required to release the (target) RNA molecules comprised therein. The extraction of a sample is required to extract the (target) RNA molecules comprised therein. The amplification of a sample is required to amplify the (target) RNA molecules comprised therein. The sequencing of a sample is required to sequence the (target) RNA molecules comprised therein.

For example, as sample a biological sample such as a whole blood sample is provided. In a first step, the sample, specifically the cells comprised in the sample, are lysed to release the RNA molecules comprised therein. Afterwards, cDNA is produced from the (target) RNA molecules using adapters ligated to the denatured (target) RNA molecules via reverse transcription. The cDNA molecules are subsequently amplified to finally allow sequencing such as next generation sequencing of said cDNA molecules.
In any of the above processes, 5′end and/or 3′end additions may be added to the (target) RNA molecules which need to be identified and (target) RNA molecules having these 5′end and/or 3′end additions have to be excluded from (RNA) data set analysis or removed from the (RNA) data set.

The sample may comprise a biological material or is derived from a biological material. Preferably, the biological material is tissue or a body fluid. More preferably, the body fluid is blood. Even more preferably, the blood is whole blood or a blood fraction. Particularly, the blood fraction is selected from the group consisting of a blood cell fraction and plasma or serum. The blood cell fraction represents the cellular portion of (whole) blood. Plasma and serum represent the acellular portion of (whole) blood. More particularly, the blood cell fraction comprises erythrocytes, leukocytes, or thrombocytes, the blood cell fraction is a fraction of erythrocytes, leukocytes, or thrombocytes, or the blood cell fraction is a mixture of erythrocytes, leukocytes, and thrombocytes.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Shows Ct values as measured by qRT-PCR were plotted against the dilution factor of mixtures containing each spike-in individually.

FIG. 2: Shows Ct values as measured by qRT-PCR were plotted against the concentration of the mixtures added to the PAXgene RNA; each spike-in individually.

FIG. 3: Shows Ct values as measured by the qPCR on cDNA samples prepared from a single RNA sample containing the mixture of all four spike-ins were plotted against the amount of the individual spike-ins added to the PAXgene sample before RNA extraction.

FIG. 4: Shows preparation of NGS library with the sample as described in FIG. 3. Plotted are raw read counts for individual spike-ins against the amount.

FIG. 5: Shows a schematic outline of the strategy for identifying non-biological, technical artefacts containing artificial affixes. In a first step, a sequencing dataset containing spike-in RNA is analysed and their affixes recorded. In a second step, these affixes can be compared with the 5′ and 3′ ends of non-spike-in sequences from either the same dataset or from a different dataset that may or may not contain the same spike-ins. Sequences that have the affixes are removed and, thus, excluded from further analysis.

FIG. 6 Shows the artificial spike-in RNAs having a nucleotide sequence according to SEQ ID NO: 1 to SEQ ID NO: 4 and the distribution of affix lengths. Higher spike-in expression leads to more findings of affixes. Furthermore, the total count of unique affixes is given and a length-specific count for lengths 1 to 18.

FIG. 7: Shows the mean expression of artificial spike-in RNAs (length: 21 nucleotides) and their longer artefacts (spike-in plus affixes of increasing length).

FIG. 8: Shows the number of removed sequences based on the minimum affix length applied for filtering. The expected error percentage gives the theoretical probability of finding the affix of a given length among all possible RNA sequences of the same length (100 (L/4L), L=RNA length). At a minimum length of 5, this probability is less than 0.5%. Increasing the minimum length reduces the number of removed sequences, while at the same time lowering the probability of falsely removing a true RNA sequence that matches the affix by coincidence. Dataset 1 consisted of clinical blood samples contained the spike-ins, dataset 2 refers to the small RNA sequencing data provided by The Cancer Genome Atlas (TCGA) and did not contain any spike-ins.

### EXAMPLES

The examples given below are for illustrative purposes only and do not limit the invention described above in any way.

### Example 1

To overcome the aforementioned caveats of using a single spike-in such as cel-miR-39-3p, a universal spike-in system that broadly reflects endogenous miRNA behaviour during RNA extraction and detection was designed and optimized.

Specifically, 20 random sequences of 21 nucleotides in length (reflecting the typical length of microRNAs) were designed (not shown). Their molecular characteristics e.g. melting temperature (Tm ° C.) and GC % were next evaluated. Afterwards, a short list of 10 artificial sequences were selected for wet lab validation on the basis of several criteria. The aim was to minimize primer dimer formation and to select RNA molecules having a relatively weak secondary structure. In addition, the RNA molecules should broadly reflect the endogenous GC content range. Accordingly, spike-ins that range from 38.1% to 61.9% GC content which encompasses the majority of endogenous microRNA GC contents were chosen.

Shortlisted artificial sequences were used to order RNA synthesis including 5′ phosphate group (IDT, Norcross, VA, USA). miRcury LNA assays (QIAgen, Venlo, The Netherlands) were ordered and uses for quantification of the spike-ins using semi-quantitative reverse-transcription PCR (qRT-PCR).

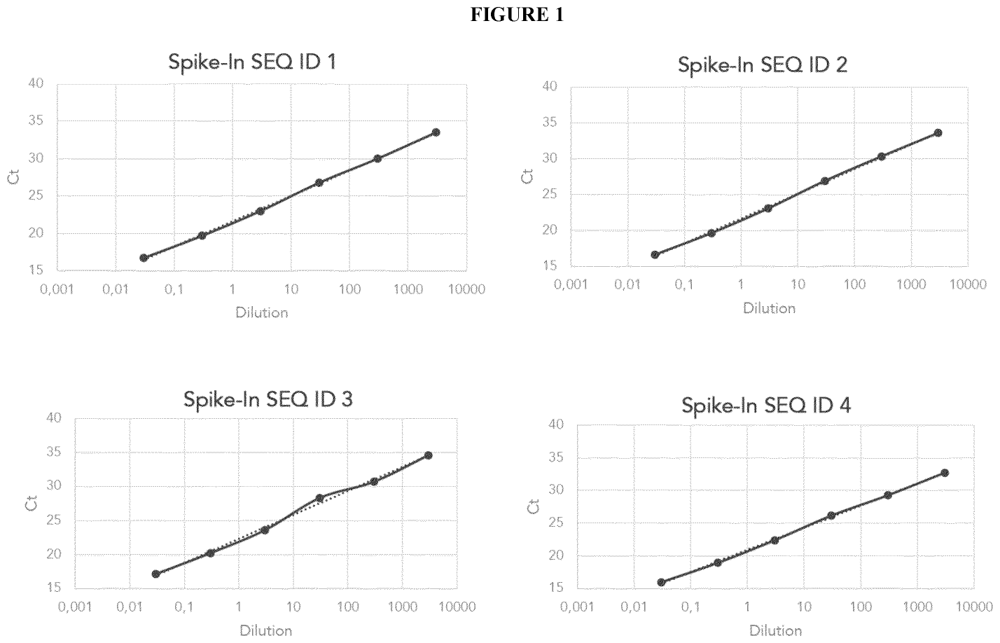

First, a ten-fold dilution series of each spike-in was prepared for each molecule separately and used for miRucry assay, and measured on Quantstudio Flex 6 (ThermoFisher Scientific, Waltham, USA). Cycle threshold (Ct) values were plotted against the dilutions factor of 6 dilutions that were used. FIG. 1 shows the data for 4 sequences (SEQ ID NO: 1 to SEQ ID NO: 4).

The spike-in with SEQ ID NO: 1 has the following nucleotide sequence: GAUAGAUACGCCAGUACCGCC,
the spike-in with SEQ ID NO: 2 has the following nucleotide sequence: AACGAAGCUCCACGAUGUAGG,
the spike-in with SEQ ID NO: 3 has the following nucleotide sequence: UGUACGGAAAUAUUGGCUACC, and
the spike-in with SEQ ID NO: 4 has the following nucleotide sequence: UUCAUACGUUGCCCAAUCCAG.

Data for other sequences are not shown.

Based on the linear regression fitting of curves from the plots showed in the FIG. 1, the slope, R-squared and primer efficiencies were calculated. These are summarized in the Table 1 below:

TABLE 1
Analysis of the data as show in the FIG. 1.

| | Slope | R squared | Primer efficiency % |
|---|---|---|---|
| SpikeIn Seq ID 1 | 3.394 | 0.9991 | 97.091 |
| SpikeIn Seq ID 2 | 3.452 | 0.9991 | 94.857 |
| SpikeIn Seq ID 3 | 3.524 | 0.9955 | 92.194 |
| SpikeIn Seq ID 1-4 | 3.380 | 0.9992 | 97.651 |

All four tested spike-in sequences showed expected slope (close to 3,33), high R-squared (above 0,99) and very good primer efficiencies (between 90-100%). Each specific miRcury LNA assay was used in a qPCR test on other non-cognate spike-in RNA, as well as on a biological PAXgene RNA sample. No amplification was observed (data not shown), suggesting that the miRcury LNA assays were specific for the cognate RNA only.

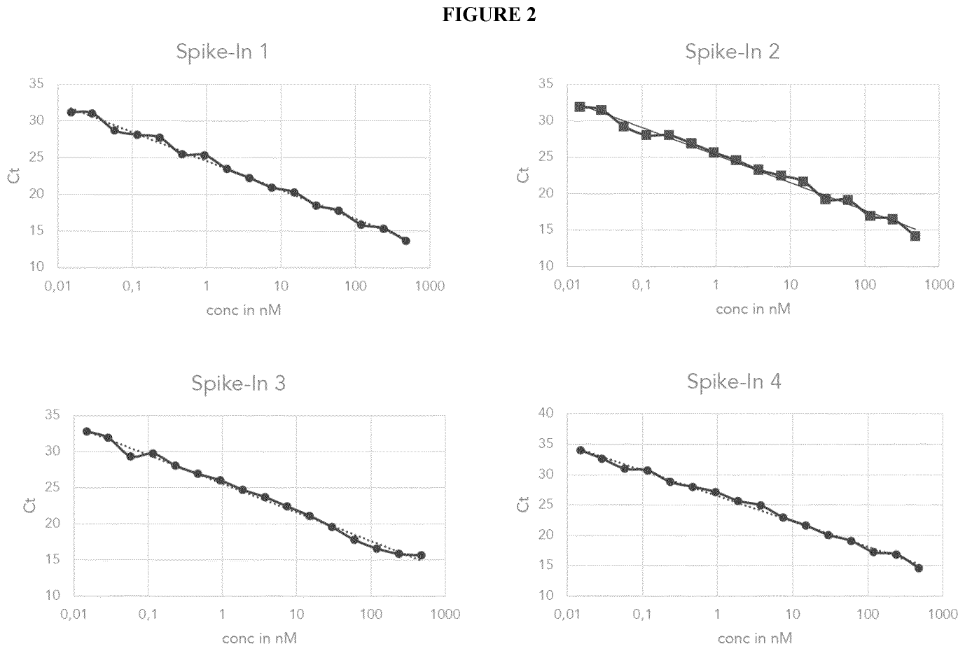

Next, each spike-in was prepared in a 2-fold dilution, added to a PAXgene RNA sample and miRcury qPCR assay was performed. FIG. 2 summarizes the data from this experiment.

R-squared of each spike-in RNA from the data as shown in FIG. 2 was calculated and is listed in the Table 2. Overall, a R-squared of 0,99 or close to ti was observed for all spike-in RNAs.

TABLE 2
Analysis of the data as shown in FIG. 2.

| | R squared |
|---|---|
| SpikeIn SEQ ID 1 | 0.9944 |
| SpikeIn SEQ ID 2 | 0.9897 |
| SpikeIn SEQ ID 3 | 0.9926 |
| SpikeIn SEQ ID 4 | 0.9949 |

Next, a test mixture of the 4 spike-ins was prepared in nuclease-free water with following concentrations of RNAs: SEQ ID NO: 1-340 pM, SEQ ID NO: 2-72.5 pM, SEQ ID NO: 3-2 pM, SEQ ID NO: 4-0.7 pM. 10 μl of this test mixture was added to the PAXgene lysate shortly before the extraction. Extraction of PAXgene was done using QIAsymphony PAXgene extraction kit (QIAgen, Venlo, The Netherlands) and eluted in 200 μl of elution buffer. This RNA was then used for the measurement of expression using qPCR. The results are summarized in the Table 3, where the comparison of predicted and measured Ct values is done. Overall, the measured Ct values are lower than the predicted, which points to a good extraction efficiency.

TABLE 3
Summary of the results from test extractions when
spike-ins were given to the PAXgene sample at given
amounts. After RNA extraction, qRT-PCR data was
obtained and compared against predicted data.

| | pM | | | Ct measured |
|---|---|---|---|---|
| Spike In | Stock | amol added | Ct predicted | (mean) |
| 1 | 340.0 | 3400 | 26.0 | 24.3 |
| 2 | 72.5 | 725 | 29.3 | 27.1 |
| 3 | 2.0 | 20 | 35.9 | 32.0 |
| 4 | 0.7 | 7 | 39.2 | 33.6 |

In a next step, the PAXgene RNA sample described in Table 3 was used for preparing a next generation sequencing library using miRNA QIAseq NGS library kit (QIAgen, Venlo, The Netherlands) as recommended by the manufacturer. Immediately after the cDNA purification step, the cDNA was diluted 1:10 and used for the qPCR reaction using spike-in specific forward primers and a common reverse primer. Specifically, the following primers were used:

SpikeIn_1_forward:
(SEQ ID NO: 5)
GATAGATACGCCAGTACCGCC,
SpikeIn_2_forward:
(SEQ ID NO: 6)
AACGAAGCTCCACGATGTAGG,
SpikeIn_3_forward:
(SEQ ID NO: 7)
TGTACGGAAATATTGGCTACC,
SpikeIn_4_forward:
(SEQ ID NO: 8)
TTCATACGTTGCCCAATCCAG,
and
SpikeIn_reverse:
(SEQ ID NO: 9)
ATGCATGCATGCATTGATGGTGCCTACAGTT.

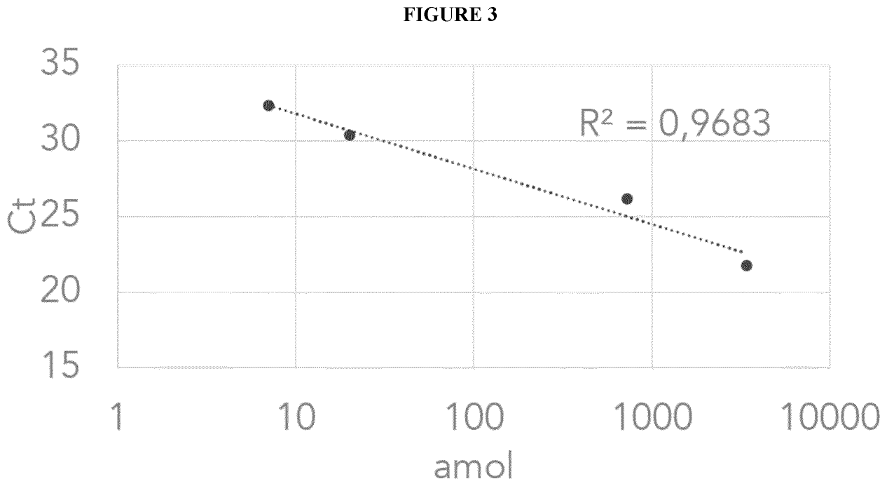

The results are listed in the Table 4, while the R-squared of the spike-ins expression as measured by the qPCR on the NGS library is shown in the FIG. 3.

TABLE 4
Analysis of the data as shown in FIG. 3.

| Spike In | pM stock | amol added | Ct measured |
|---|---|---|---|
| 1 | 340.0 | 3400 | 21.74 |
| 2 | 72.5 | 725 | 26.16 |
| 3 | 2.0 | 20 | 30.37 |
| 4 | 0.7 | 7 | 32.32 |

In conclusion, an acceptable linearity (>0.95) as measured on the cDNA from library preparation process was achieved.

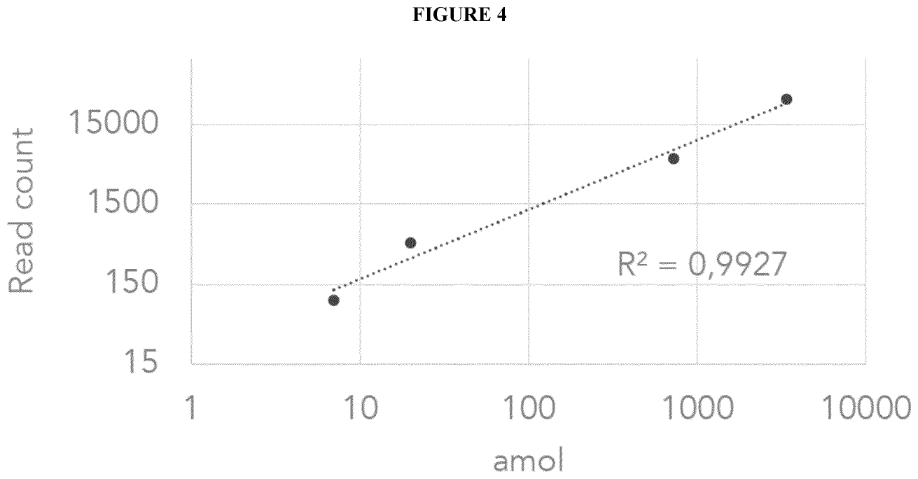

Finally, the cDNA samples from the previous experiment were used for library PCR and NGS. After data pre-processing, raw read counts to the respective spike ins (Table 5) were assigned. It was observed that also on the level of NGS read counts the spike-ins are expressed in expected linear order (FIG. 4).

TABLE 5
Analysis of the data as shown in FIG. 4.

| Spike In | pM stock | amol added | Read counts |
|---|---|---|---|
| 1 | 340.0 | 3400 | 30628 |
| 2 | 72.5 | 725 | 5554 |
| 3 | 2.0 | 20 | 491 |
| 4 | 0.7 | 7 | 94 |

### Example 2

The above-mentioned artificial spike-in RNAs (having a nucleotide sequence according to SEQ ID NO: 1 to SEQ ID NO: 4) were further used to identify artificial target RNAs produced by the next generation sequencing (NGS) technology. Specifically, target RNAs with 5′end and/or 3′end attachments were identified. During NGS, parts of other RNAs were found attached to the spike-in RNAs on both ends (5′ and 3′). These parts are here called affixes, which include both prefixes (5′) and suffixes (3′). Adapter sequences of adapters used in the NGS process were also found to be attached to the spike-in RNAs.

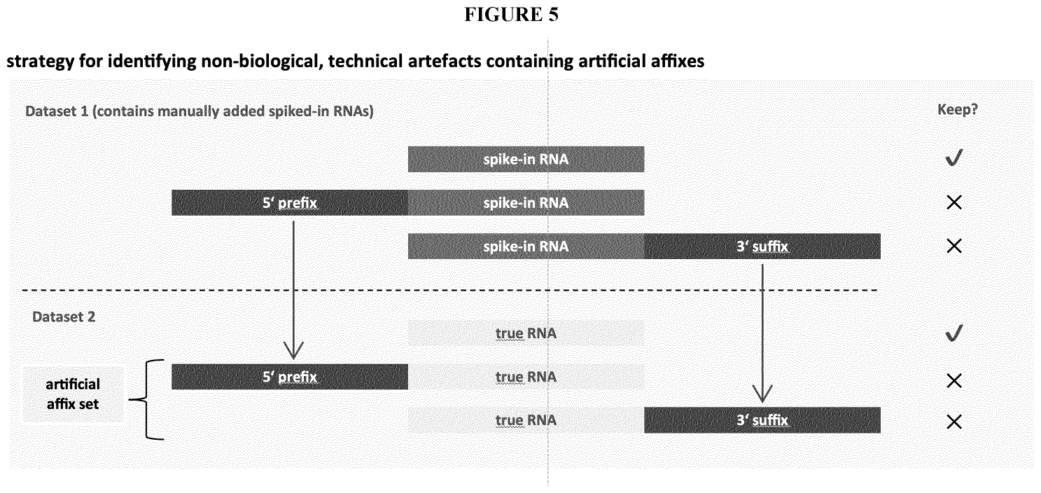

The internal (HBDX) lung disease dataset (blood samples) of the present inventors as well as the external TCGA dataset (tissue samples from cancer patients) were used. The HBDX dataset contained the spike-ins and was used to identify affixes. The same affixes were found in TCGA as well (at a lower rate), despite no spike-ins being added. In part, the affixes could be linked to 5′-adapter sequence GTTCAGAGTTCTACAGTCCGACGATC (SEQ ID NO: 84) commonly used during small-RNA NGS. In this respect, we like to refer to FIG. 5.

With the identified affixes attached to the spike-in RNAs it was possible to also identify target RNAs that are likely artificial/modified, i.e. they were produced by the same “affix-attaching” mechanism as the spike-ins themselves. As these target RNAs can be misleading during data analysis, they should be removed to improve data set quality and subsequent analysis.

To prevent the false removal of target RNAs, only target RNAs with a minimum affix length of 5 nucleotides or more were removed from subsequent analysis/the data set.

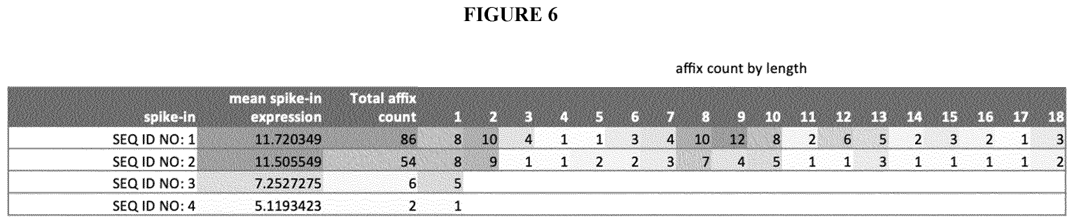

FIG. 6 shows the spike-in RNAs having a nucleotide sequence according to SEQ ID NO: 1 to SEQ ID NO: 4 and the distribution of affix lengths. Higher spike-in expression leads to more findings of affixes.

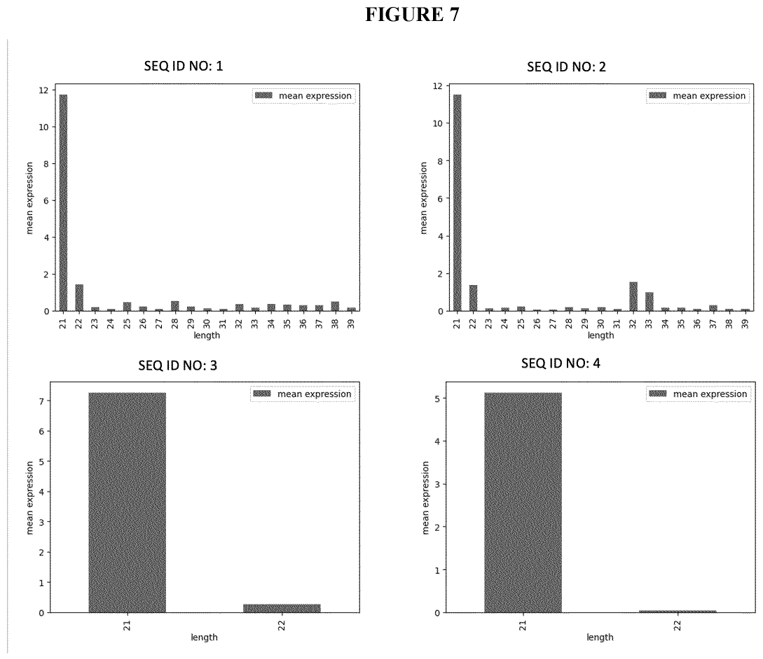

FIG. 7 shows the mean expression of spike-in RNAs (length: 21 nucleotides) and their longer artefacts (spike-in plus affixes of increasing length).

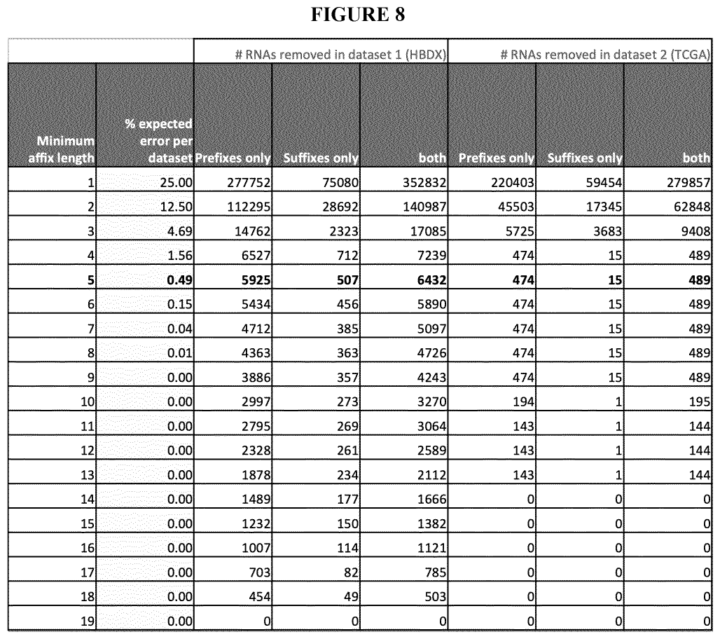

FIG. 8 shows how many sequences would be removed in the two datasets (HBDX, TCGA) if only affixes of a minimum length (or longer) were considered. A theoretical guideline is given as a percentage of coincidental match among all possible nucleotide sequences of the same length as the affix. The lower this theoretical error percentage should be, the higher the minimum length must be chosen. However, at a minimum length of 5 the error percentage drops below 0.5%, which may be acceptable in most settings.

1. A composition comprising at least 3 RNA molecules, wherein the at least 3 RNA molecules are selected from a group consisting of RNA molecules having a nucleotide sequence according to SEQ ID NO: 1 to SEQ ID NO: 4, a fragment thereof, and a sequence having at least 80% sequence identity thereto.

2. (canceled)

3. The composition of claim 1, wherein the composition comprises RNA molecules having a nucleotide sequence according to
(i) SEQ ID NO: 1, SEQ ID NO: 2, and SEQ ID NO: 3,
(ii) SEQ ID NO: 2, SEQ ID NO: 3, and SEQ ID NO: 4,
(iii) SEQ ID NO: 1, SEQ ID NO: 3, and SEQ ID NO: 4,
(iv) SEQ ID NO: 1, SEQ ID NO: 2, and SEQ ID NO: 4, or
(v) SEQ ID NO: 1, SEQ ID NO: 2, SEQ ID NO: 3, and SEQ ID NO: 4.

4-5. (canceled)

6. The composition of claim 1, wherein the at least 3 RNA molecules are comprised in the composition in a gradient of defined amounts.

7-8. (canceled)

9. The composition of claim 1, wherein the at least 3 RNA molecules comprised in the composition are present in an amount of between 0.1 amol to 4000 amol, and even more preferably in an amount of between 1 amol and 3500 amol.

10. The composition of claim 9, wherein the 4 RNA molecules comprised in the composition are present in an amount of between 1 amol and 3500 amol.

11. The composition of claim 1, wherein the composition comprises at least 3 RNA molecules and wherein
the first RNA molecule is comprised in an amount of about 3400 amol, the second RNA molecule is comprised in an amount of about 725 amol, and the third RNA molecule is comprised in an amount of about 20 amol, or
the first RNA molecule is comprised in an amount of about 1360 amol, the second RNA molecule is comprised in an amount of about 290 amol, and the third RNA molecule is comprised in an amount of about 80 amol.

12. The composition of claim 11, wherein the composition comprises 4 RNA molecules and wherein
the first RNA molecule is comprised in an amount of about 3400 amol, the second RNA molecule is comprised in an amount of about 725 amol, the third RNA molecule is comprised in an amount of about 20 amol, and the fourth RNA molecule is comprised in an amount of about 7 amol, or
the first RNA molecule is comprised in an amount of about 1360 amol, the second RNA molecule is comprised in an amount of about 290 amol, the third RNA molecule is comprised in an amount of about 80 amol, and the fourth RNA molecule is comprised in an amount of about 27 amol.

13-19. (canceled)

20. A kit comprising the composition of claim 1.

21-24. (canceled)

25. A method for examining a sample comprising the step of:
evaluating a sample with respect to the at least 3 RNA molecules comprised in/from the composition of claim 1.

26-33. (canceled)

34. The method of claim 25, wherein the evaluation comprises determining whether the at least 3 RNA molecules show a characteristic distribution.

35. The method of claim 34, wherein, if the characteristic distribution is given, the sample is further processed and/or analysed.

36. The method of claim 35, wherein the characteristic distribution is given if
the at least 3 RNA molecules are present in their expected level,
the at least 3 RNA molecules are present at their expected order/rank (defined by the relation of the levels, in particular amounts, of the at least 3 RNA molecules), and/or
the at least 3 RNA molecules are present in their expected linearity.

37. The method of claim 36, wherein the expected order results in a Spearman's rank correlation coefficient (Spearman's ρ) of ≥0.95 and/or the expected linearity results in a Pearson's correlation coefficient (Pearson's r) of ≥ 0.66.

38-46. (canceled)

47. The method of claim 25, wherein the evaluation comprises identifying 5′end and/or 3′end additions of the at least 3 RNA molecules comprised in/from the composition of claim 1.

48-49. (canceled)

50. The method of claim 47, wherein the 5′end and/or 3′ end additions are the result of RNA molecule/adapter fusion, RNA molecule/RNA molecule fusion, adapter/adapter fusion.

51. The method of claim 25, wherein the sample comprises target RNA molecules and wherein the presence of 5′end and/or 3′end additions identified with respect to the at least 3 RNA molecules comprised in/from the composition of claims 1 is indicative for the presence of 5′end and/or 3′end additions in the target RNA molecules.

52. (canceled)

53. The method of claim 51, wherein target RNA molecules with 5′end and/or 3′end additions comprised in the sample are excluded from further analyses/are not further used.

54-56. (canceled)

57. A method for
(i) optimized processing of biological samples,
(ii) optimized RNA preparation from or RNA analysis of biological samples, or
(iii) improving RNA data set quality comprising the step of:
carrying out the method of claim 25.

58-59. (canceled)

60. A method for improving RNA data set quality comprising the steps of:
(i) determining the sequence of RNA molecules in a sample,
(ii) determining 5′end and/or 3′end additions to the RNA molecules, which are not part of the RNA molecules in naturally occurring form, and
(iii) excluding RNA molecules having 5′end and/or 3′end additions from RNA data set analysis/removing RNA molecules having 5′end and/or 3′end additions from the RNA data set.

61. The method of claim 60, wherein the determination of the sequence of RNA molecules in the sample encompasses:
denaturation of RNA molecules and ligation of 5′adapters and/or 3′adapters to the denatured RNA molecules,
reverse transcription of RNA molecules (having 5′adapters and/or 3′adapters ligated thereon) into cDNA molecules,
amplification of (said) cDNA molecules, and/or
sequencing, preferably next generation sequencing, of (said) cDNA molecules.

62-68. (canceled)


SEQUENCE LISTING (103 sequences)

SEQ ID NO: 1 - RNA, 21 nt, synthetic construct
  source 1..21: mol_type = other RNA
gatagatacgccagtaccgcc

SEQ ID NO: 2 - RNA, 21 nt, synthetic construct
  source 1..21: mol_type = other RNA
aacgaagctccacgatgtagg

SEQ ID NO: 3 - RNA, 21 nt, synthetic construct
  source 1..21: mol_type = other RNA
tgtacggaaatattggctacc

SEQ ID NO: 4 - RNA, 21 nt, synthetic construct
  source 1..21: mol_type = other RNA
ttcatacgttgcccaatccag

SEQ ID NO: 5 - DNA, 21 bp, synthetic construct
  source 1..21: mol_type = other DNA
gatagatacgccagtaccgcc

SEQ ID NO: 6 - DNA, 21 bp, synthetic construct
  source 1..21: mol_type = other DNA
aacgaagctccacgatgtagg

SEQ ID NO: 7 - DNA, 21 bp, synthetic construct
  source 1..21: mol_type = other DNA
tgtacggaaatattggctacc

SEQ ID NO: 8 - DNA, 21 bp, synthetic construct
  source 1..21: mol_type = other DNA
ttcatacgttgcccaatccag

SEQ ID NO: 9 - DNA, 31 bp, synthetic construct
  source 1..31: mol_type = other DNA
atgcatgcatgcattgatggtgcctacagtt

SEQ ID NO: 10-46 - skipped (37 entries)

SEQ ID NO: 47 - DNA, 10 bp, synthetic construct
  source 1..10: mol_type = other DNA
cggagggcgg

SEQ ID NO: 48 - DNA, 10 bp, synthetic construct
  source 1..10: mol_type = other DNA
gtccgcgatc

SEQ ID NO: 49 - DNA, 10 bp, synthetic construct
  source 1..10: mol_type = other DNA
gtcgacgatc

SEQ ID NO: 50 - DNA, 10 bp, synthetic construct
  source 1..10: mol_type = other DNA
cgggcggatc

SEQ ID NO: 51 - DNA, 10 bp, synthetic construct
  source 1..10: mol_type = other DNA
tggaggcgtg

SEQ ID NO: 52 - DNA, 10 bp, synthetic construct
  source 1..10: mol_type = other DNA
tccgacgatc

SEQ ID NO: 53 - DNA, 10 bp, synthetic construct
  source 1..10: mol_type = other DNA
ggggcgtggg

SEQ ID NO: 54 - DNA, 10 bp, synthetic construct
  source 1..10: mol_type = other DNA
aagcggggct

SEQ ID NO: 55 - DNA, 10 bp, synthetic construct
  source 1..10: mol_type = other DNA
cggggagcca

SEQ ID NO: 56 - DNA, 11 bp, synthetic construct
  source 1..11: mol_type = other DNA
gtccgacgatc

SEQ ID NO: 57 - DNA, 11 bp, synthetic construct
  source 1..11: mol_type = other DNA
tcggagggcgg

SEQ ID NO: 58 - DNA, 12 bp, synthetic construct
  source 1..12: mol_type = other DNA
agtccgacgatc

SEQ ID NO: 59 - DNA, 12 bp, synthetic construct
  source 1..12: mol_type = other DNA
aagcggggctgg

SEQ ID NO: 60 - DNA, 12 bp, synthetic construct
  source 1..12: mol_type = other DNA
gtccgacggatc

SEQ ID NO: 61 - DNA, 12 bp, synthetic construct
  source 1..12: mol_type = other DNA
tcgggctggggc

SEQ ID NO: 62 - DNA, 12 bp, synthetic construct
  source 1..12: mol_type = other DNA
tacctggttgat

SEQ ID NO: 63 - DNA, 12 bp, synthetic construct
  source 1..12: mol_type = other DNA
tcggggcggcgg

SEQ ID NO: 64 - DNA, 13 bp, synthetic construct
  source 1..13: mol_type = other DNA
cagtccgacgatc

SEQ ID NO: 65 - DNA, 13 bp, synthetic construct
  source 1..13: mol_type = other DNA
tacctggttgatc

SEQ ID NO: 66 - DNA, 13 bp, synthetic construct
  source 1..13: mol_type = other DNA
tcgggctggggcg

SEQ ID NO: 67 - DNA, 13 bp, synthetic construct
  source 1..13: mol_type = other DNA
tggaggcgtgggt

SEQ ID NO: 68 - DNA, 14 bp, synthetic construct
  source 1..14: mol_type = other DNA
acagtccgacgatc

SEQ ID NO: 69 - DNA, 14 bp, synthetic construct
  source 1..14: mol_type = other DNA
ggtcgggctggggc

SEQ ID NO: 70 - DNA, 15 bp, synthetic construct
  source 1..15: mol_type = other DNA
cggaagcgtgctggg

SEQ ID NO: 71 - DNA, 15 bp, synthetic construct
  source 1..15: mol_type = other DNA
ggtcgggctggggcg

SEQ ID NO: 72 - DNA, 15 bp, synthetic construct
  source 1..15: mol_type = other DNA
tacagtccgacgatc

SEQ ID NO: 73 - DNA, 16 bp, synthetic construct
  source 1..16: mol_type = other DNA
cggaagcgtgctgggc

SEQ ID NO: 74 - DNA, 16 bp, synthetic construct
  source 1..16: mol_type = other DNA
ctacagtccgacgatc

SEQ ID NO: 75 - DNA, 17 bp, synthetic construct
  source 1..17: mol_type = other DNA
tctacagtccgacgatc

SEQ ID NO: 76 - DNA, 18 bp, synthetic construct
  source 1..18: mol_type = other DNA
cggaagcgtgctgggccc

SEQ ID NO: 77 - DNA, 18 bp, synthetic construct
  source 1..18: mol_type = other DNA
tcggggcggcggcggcgg

SEQ ID NO: 78 - DNA, 18 bp, synthetic construct
  source 1..18: mol_type = other DNA
ttctacagtccgacgatc

SEQ ID NO: 79 - DNA, 18 bp, synthetic construct
  source 1..18: mol_type = other DNA
tagcagcacatcatggtt

SEQ ID NO: 80 - skipped

SEQ ID NO: 81 - DNA, 10 bp, synthetic construct
  source 1..10: mol_type = other DNA
ggggcgtggg

SEQ ID NO: 82 - DNA, 13 bp, synthetic construct
  source 1..13: mol_type = other DNA
tggaggcgtgggt

SEQ ID NO: 83 - DNA, 21 bp, synthetic construct
  source 1..21: mol_type = other DNA
tggaattctcgggtgccaagg

SEQ ID NO: 84 - DNA, 26 bp, synthetic construct
  source 1..26: mol_type = other DNA
gttcagagttctacagtccgacgatc

SEQ ID NO: 85 - DNA, 21 bp, synthetic construct
  source 1..21: mol_type = other DNA
tggaattctcgggtgccaagg

SEQ ID NO: 86 - DNA, 22 bp, synthetic construct
  source 1..22: mol_type = other DNA
gaattccaccacgttcccgtgg

SEQ ID NO: 87 - DNA, 50 bp, synthetic construct
  source 1..50: mol_type = other DNA
aatgatacggcgaccaccgagatctacacgttcagagttctacagtccga

SEQ ID NO: 88 - DNA, 24 bp, synthetic construct
  source 1..24: mol_type = other DNA
caagcagaagacggcatacgagat

SEQ ID NO: 89 - DNA, 33 bp, synthetic construct
  source 1..33: mol_type = other DNA
gtgactggagttccttggcacccgagaattcca

SEQ ID NO: 90 - DNA, 21 bp, synthetic construct
  source 1..21: mol_type = other DNA
caagcagaagacggcatacga

SEQ ID NO: 91 - DNA, 26 bp, synthetic construct
  source 1..26: mol_type = other DNA
gttcagagttctacagtccgacgatc

SEQ ID NO: 92 - DNA, 22 bp, synthetic construct
  source 1..22: mol_type = other DNA
tcgtatgccgtcttctgcttgt

SEQ ID NO: 93 - DNA, 24 bp, synthetic construct
  source 1..24: mol_type = other DNA
atctcgtatgccgtcttctgcttg

SEQ ID NO: 94 - DNA, 21 bp, synthetic construct
  source 1..21: mol_type = other DNA
caagcagaagacggcatacga

SEQ ID NO: 95 - DNA, 44 bp, synthetic construct
  source 1..44: mol_type = other DNA
aatgatacggcgaccaccgacaggttcagagttctacagtccga

SEQ ID NO: 96 - DNA, 32 bp, synthetic construct
  source 1..32: mol_type = other DNA
cgacaggttcagagttctacagtccgacgatc

SEQ ID NO: 97 - DNA, 46 bp, synthetic construct
  source 1..46: mol_type = other DNA
aaaaaaaaaaaaaaaaaaaaaaaaaaaaaaaaaaaaaaaaaaaaaa

SEQ ID NO: 98 - DNA, 33 bp, synthetic construct
  source 1..33: mol_type = other DNA
agatcggaagagcacacgtctgaactccagtca

SEQ ID NO: 99 - DNA, 33 bp, synthetic construct
  source 1..33: mol_type = other DNA
agatcggaagagcgtcgtgtagggaaagagtgt

SEQ ID NO: 100 - DNA, 33 bp, synthetic construct
  source 1..33: mol_type = other DNA
gatcggaagagcacacgtctgaactccagtcac

SEQ ID NO: 101 - DNA, 24 bp, synthetic construct
  source 1..24: mol_type = other DNA
atctcgtatgccgtcttctgcttg

SEQ ID NO: 102 - DNA, 29 bp, synthetic construct
  source 1..29: mol_type = other DNA
aatgatacggcgaccaccgagatctacac

SEQ ID NO: 103 - DNA, 33 bp, synthetic construct
  source 1..33: mol_type = other DNA
acactctttccctacacgacgctcttccgatct